(19) United States
(12) Patent Application Publication
(10) Pub. No.: US 2018/0177437 A1
(43) Pub. Date: Jun. 28, 2018

(54) BIOLOGICAL INFORMATION MEASURING DEVICE, WEARABLE DEVICE, AND SENSOR INFORMATION PROCESSING DEVICE

(71) Applicant: SEIKO EPSON CORPORATION, Tokyo (JP)

(72) Inventor: Hiroki YOSHIOKA, Matsumoto-shi (JP)

(73) Assignee: SEIKO EPSON CORPORATION, Tokyo (JP)

(21) Appl. No.: 15/854,463

(22) Filed: Dec. 26, 2017

(30) Foreign Application Priority Data
Dec. 26, 2016 (JP) 2016-251101
Nov. 6, 2017 (JP) 2017-213510

(51) Int. Cl. A61B 5/11; A61B 5/00; G06F 1/10

(52) U.S. Cl. CPC A61B 5/1118; A61B 5/681; A61B 5/0015; G04G 3/00; G06F 1/10; A61B 2560/0242; A61B 5/742

(57) ABSTRACT

A biological information measuring device includes a first processor including a first interface configured to acquire a biological signal output from a biological sensor and a second interface configured to acquire a satellite signal and a second processor configured to control at least one of a display section and a communication section and electrically connected to the first processor.

This application claims the benefit of Japanese Application No. JP 2016-251101 filed Dec. 26, 2016, and Japanese Application No. JP 2017-213510 filed Nov. 6, 2017. The disclosure of the prior applications is hereby incorporated by reference herein in their entirety.

### BACKGROUND

### 1. Technical Field

The present invention relates to a biological information measuring device, a wearable device, a sensor information processing device, and the like.

### 2. Related Art

There has been widely known a biological information measuring device including a GPS (Global Positioning System) and a biological sensor. The biological information measuring device is realized as, for example, a wearable device worn on a user.

For example, JP-A-2005-195425 (Patent Literature 1) discloses an ear-mounted electronic device (a headphone-type device) including means for measuring a position on the basis of signals transmitted from GPS satellites and means for detecting biological information.

In a use in which measurement needs to be performed for a long time such as life log measurement, it is important to set a measurable period per charging as long as possible. When a biological information measuring device is a wearable device, in order to reduce a user load due to wearing of the wearable device, a reduction in the size and a reduction in the weight of the wearable device are important. There is often a limit in a battery capacity. Therefore, in the biological information measuring device, it is extremely important to reduce power consumption to enable the biological information measuring device to operate efficiently with limited electric power.

However, the biological information measuring device in the past often adopts a form in which a CPU, which controls the entire device, processes measurement data. Power consumption of the CPU, which controls the entire device, is large. This is a problem in terms of a reduction in power consumption. Patent Literature 1 does not disclose configurations of a processor and an interface for reducing power consumption.

### SUMMARY

An advantage of some aspects of the invention is to provide a biological information measuring device, a wearable device, a sensor information processing device, and the like capable of reducing power consumption with an efficient configuration.

An aspect of the invention relates to a biological information measuring device including: a first processor including a first interface configured to acquire a biological signal output from a biological sensor and a second interface configured to acquire a satellite signal; and a second processor configured to control at least one of a display section and a communication section and electrically connected to the first processor.

In the aspect of the invention, the first processor is capable of acquiring the biological signal and the satellite signal. Therefore, the biological signal and the satellite signal can be treated by a processor different from a processor (in a narrow sense, a host CPU) that performs control of sections of the biological information measuring device. Therefore, it is possible to, for example, reduce power consumption.

In the biological information measuring device according to the aspect of the invention, the first processor may include a processing section connected to the first interface and the second interface and configured to perform processing based on the biological signal and the satellite signal.

With this configuration, the first processor is capable of performing the processing for the biological signal and the satellite signal.

In the biological information measuring device according to the aspect of the invention, the first processor may be capable of operating in any one operation mode of a plurality of operation modes, and the processing section may perform, on the basis of the biological signal, processing for switching the operation mode.

With this configuration, it is possible to, for example, operate the first processor in an appropriate operation mode conforming to the biological signal.

In the biological information measuring device according to the aspect of the invention, the first processor may include a third interface configured to acquire a body motion signal output from a body motion sensor, and the processing section may perform, on the basis of at least one of the biological signal and the body motion signal, the processing for switching the operation mode.

With this configuration, it is possible to, for example, operate the first processor in an appropriate operation mode conforming to at least one of the biological signal and the body motion signal.

In the biological information measuring device according to the aspect of the invention, the first processor may include a nonvolatile memory configured to store a plurality of operation programs corresponding to the respective plurality of operation modes.

With this configuration, it is possible to, for example, flexibly switch the operation mode (an operation program) using the nonvolatile memory.

In the biological information measuring device according to the aspect of the invention, the processing section, the first interface, and the second interface may be formed on a semiconductor chip of one chip, and the nonvolatile memory may be stacked on the semiconductor chip.

With this configuration, it is possible to, for example, package the first processor including a semiconductor memory.

In the biological information measuring device according to the aspect of the invention, the plurality of operation modes may include at least two of a clock display mode, an activity meter mode, and a workout mode.

With this configuration, it is possible to realize the biological information measuring device that operates in a predetermined operation mode.

In the biological information measuring device according to the aspect of the invention, the first processor may include a fourth interface configured to acquire an environment signal output from an environment sensor.

With this configuration, it is possible to, for example, acquire and process an environment signal representing a state of a peripheral environment.

In the biological information measuring device according to the aspect of the invention, the first processor may further include a clock supply section configured to receive an input of a plurality of clock signals and supply a selected clock signal among the plurality of clock signals, and the clock supply section may select, on the basis of the biological signal, the clock signal to be supplied.

With this configuration, it is possible to, for example, reduce power consumption by changing the clock signal according to a situation and use the biological signal for the selection of the clock signal.

In the biological information measuring device according to the aspect of the invention, the clock supply section may perform selection of the clock signal supplied at a start time and selection of the clock signal supplied at an operation time after the start.

With this configuration, it is possible to perform clock signal control during the start and clock signal control during the operation after the start.

In the biological information measuring device according to the aspect of the invention, the biological information measuring device may further include an external oscillator provided on an outside of the first processor, the first processor may include an internal oscillator, and the clock signals may include a signal output from the external oscillator and a signal output from the internal oscillator.

With this configuration, it is possible to, for example, supply an appropriate clock signal on the basis of signals output from different oscillators.

In the biological information measuring device according to the aspect of the invention, the first processor may include a clock-frequency control section configured to change, on the basis of at least one of the biological signal, the satellite signals, and an environment signal from an environment sensor, a clock frequency of a clock signal supplied to the processing section.

With this configuration, by performing the control of the clock frequency using the biological signal, the satellite signals, the environment signal, or the like, it is possible to appropriately change the clock frequency according to a situation and reduce power consumption.

In the biological information measuring device according to the aspect of the invention, the first processor may include an internal oscillator configured to generate the clock signal, and the clock-frequency control section may change the clock frequency by controlling an oscillation frequency of the internal oscillator.

With this configuration, it is possible to realize a change of the frequency of the clock signal according to the control of the internal oscillator.

In the biological information measuring device according to the aspect of the invention, the internal oscillator may include a switching-timing control section configured to perform, when the clock frequency is changed, switching timing control for suppressing glitch occurrence in the clock signal.

With this configuration, it is possible to realize the switching of the frequency with easy control and reduce power consumption involved in the frequency switching.

Another aspect of the invention relates to a wearable device including the biological information measuring device described above.

Another aspect of the invention relates to a sensor information processing device including: a processing section; an interface configured to acquire a biological signal output from a biological sensor; a nonvolatile memory configured to store a plurality of operation programs corresponding to a plurality of operation modes; and a storing section. The operation program is a computer program that operates on the basis of sensor information output from the biological sensor. An operation program selected out of the plurality of operation programs is loaded to the storing section. The processing section operates according to the operation program loaded to the storing section.

In the another aspect of the invention, the plurality of operation programs are stored in the nonvolatile memory. The processing section is operated by loading a selected operation program among the plurality of operation programs to the storing section. Consequently, it is possible to, for example, realize a highly versatile sensor information processing device capable of operating with an appropriate operation program according to a situation and capable of processing the biological signal.

In the sensor information processing device according to the other aspect of the invention, the processing section may perform, according to the operation program loaded to the storing section, processing for changing a clock frequency of a clock signal supplied to the processing section.

With this configuration, it is possible to operate the processing section at an appropriate clock frequency conforming to the operation program.

### BRIEF DESCRIPTION OF THE DRAWINGS

The invention will be described with reference to the accompanying drawings, wherein like numbers reference like elements.

FIG. 1 is a configuration example of a biological information measuring device.

FIG. 2 is a configuration example of a package of the biological information measuring device.

FIG. 3 is a connection example of a first processor and a biological sensor.

FIG. 4 is a connection example of the first processor and the biological sensor.

FIG. 5 is an exterior view of a wearable device.

FIG. 6 is an exterior view of the wearable device.

FIG. 7 is an explanatory diagram of operation modes and processing contents.

FIG. 8 is an explanatory diagram of a software configuration.

FIG. 9 is an explanatory diagram of the software configuration.

FIG. 10 is a configuration example of a clock supply section.

FIG. 11 is a flowchart for explaining processing in an embodiment.

FIG. 12 is a flowchart for explaining application end processing.

FIG. 13 is a flowchart for explaining application start processing.

FIG. 14 is an explanatory diagram of an operation program retaining format in a nonvolatile memory.

FIG. 15 is a configuration example of an internal oscillator.

FIG. 16 is another configuration example of the internal oscillator.

FIG. 17 is a waveform chart for explaining that a glitch occurs because of switching of a frequency.

FIG. 18 is a waveform chart for explaining a method in the embodiment.

### DESCRIPTION OF EXEMPLARY EMBODIMENTS

Embodiments are explained below. Note that the embodiments explained below do not unduly limit contents of the invention described in the appended claims. All components explained in the embodiments are not always essential constituent elements of the invention.

### 1. System Configuration Example

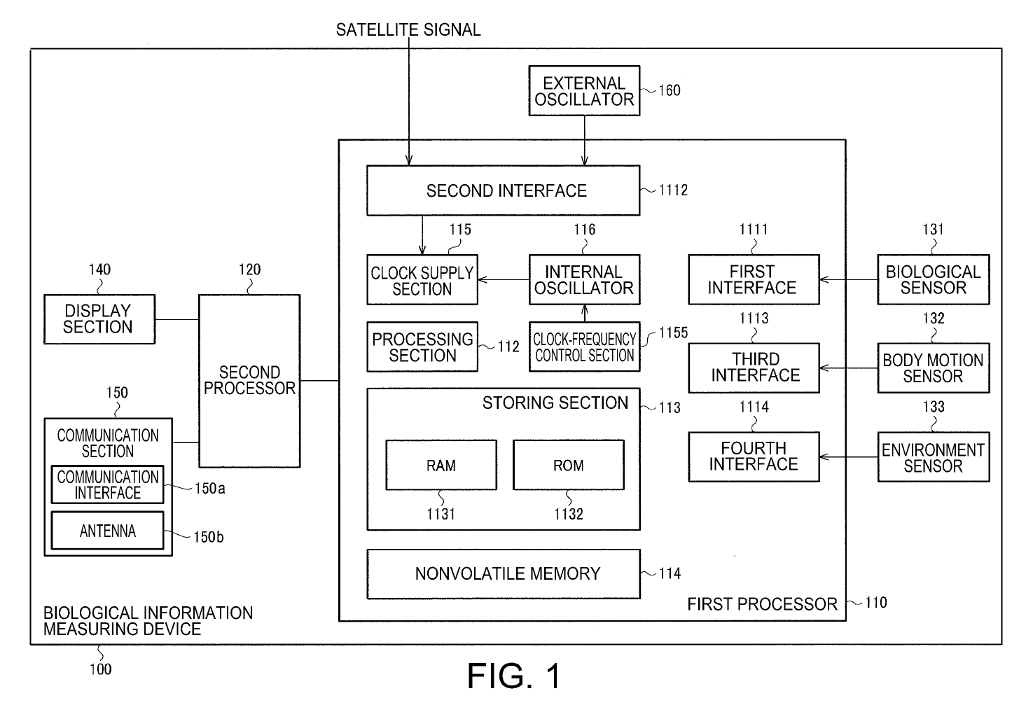

FIG. 1 is a configuration example of a biological information measuring device 100 (a performance monitoring device) in the embodiment. The biological information measuring device 100 includes a first processor 110, a second processor 120, a biological sensor 131, a body motion sensor 132, an environment sensor 133, a display section 140 (a display), a communication section 150 (a communication interface 150a and an antenna 150b), and an external oscillator 160 (a TCXO). However, the biological information measuring device 100 and sections of the biological information measuring device 100 are not limited to the components shown in FIG. 1. Various modified implementations are possible to, for example, omit a part of the components and add other components.

The first processor 110 includes, as shown in FIG. 1, first to fourth interfaces 1111 to 1114, a processing section 112 (a processor), a storing section 113 (a memory), a nonvolatile memory 114, a clock supply section 115 (a clock supply circuit and a clock-signal selection circuit), a clock-frequency control section 1155 (a clock-frequency control circuit and a control-signal generation circuit), and an internal oscillator 116. The storing section 113 includes a RAM (Random Access Memory) 1131 and a ROM (Read Only Memory) 1132.

The first interface 1111 acquires a biological signal output from the biological sensor 131. The second interface 1112 acquires a satellite signal. The third interface 1113 acquires a body motion signal output from the body motion sensor 132. The fourth interface 1114 acquires an environment signal output from the environment sensor 133.

The first interface 1111, the third interface 1113, and the fourth interface 1114 are interfaces with various sensors set as processing targets in the biological information measuring device 100 and can be realized by serial interfaces (serial buses) such as an 12C and an SPI (Serial Peripheral Interface). However, various interfaces different from the I2C and the SPI can be applied to the interfaces.

The first interface 1111, the third interface 1113, and the fourth interface 1114 can also be provided in common rather than being respectively provided as different components. For example, an I2C including a plurality of communication channels may be provided and the communication channels may be capable of operating as all of the first interface 1111, the third interface 1113, and the fourth interface 1114.

The satellite signal may be a signal transmitted from a positioning satellite, that is, a GNSS (Global Navigation Satellite System) signal. In this case, the second interface 1112 includes an antenna for the GNSS, a part of a circuit that processes a signal received by the antenna (in a narrow sense, an RF circuit), and an interface for communicating the satellite signal in a substrate. Note that the GNSS may be a GPS (Global Positioning System), may be a Galileo, may be a GLONASS (Global Navigation Satellite System), or may be other systems. The GNSS may be a system obtained by developing these systems or a system that supports these systems, for example, a quasi-zenith satellite.

The processing section 112 is connected to the first interface 1111 and the second interface 1112 and performs processing based on the biological signal and the satellite signal. The processing section 112 is a core of the first processor 110 and can be realized by, for example, a CPU (Central Processing Unit), a DSP (Digital Signal Processor), and a GPU (Graphics Processing Unit). The processing section 112 may include a hardware circuit by an ASIC (Application Specific Integrated Circuit). The processing section 112 may include an amplifier circuit, a filter circuit, and the like that process an analog signal.

The storing section 113 functions as a work area of the processing section 112 and the like. The function of the storing section 113 can be realized by a memory such as a RAM, a HDD (Hard Disk Drive), and the like. As shown in FIG. 1, the storing section 113 may include the RAM 1131 and the ROM 1132. The storing section 113 (the RAM 1131) may store a sensor signal output from a sensor such as the biological sensor 131 and information acquired as a result of some kind of processing performed by the processing section 112 on the sensor signal.

The nonvolatile memory 114 is a memory that stores a plurality of operation programs and can be realized by semiconductor memories such as an EEPROM (Electrically Erasable Programmable Read-Only Memory) and a flash memory, a HDD, and the like. In the following explanation, an example is explained in which the nonvolatile memory 114 is a flash memory.

The clock supply section 115 receives an input of a plurality of clock signals and supplies a selected clock signal among the plurality of clock signals. Specifically, the clock supply section 115 supplies, to the sections of the first processor 110, a clock signal based on at least one of the external oscillator 160 provided on the outside of the first processor 110 and the internal oscillator 116 provided on the inside of the first processor 110.

The external oscillator 160 may be, for example, an oscillator used for processing (frequency conversion) of a GNSS signal in the RF circuit. Specifically, a TCXO (Temperature Compensated Crystal Oscillator) having high accuracy is used as the external oscillator 160. The internal oscillator 116 is, for example, a ring oscillator. However, oscillators (oscillation circuits) of other forms are not prevented from being used as the external oscillator 160 and the internal oscillator 116.

The clock-frequency control section 1155 performs, on the basis of at least one of a biological signal, a satellite signal, and an environment signal, control for changing a clock frequency of a clock signal supplied to the processing section 112. In a narrow sense, the clock-frequency control section 1155 performs control for changing an oscillation frequency of the internal oscillator 116.

The biological information measuring device 100 (the first processor 110) may include a not-shown baseband circuit. The baseband circuit is a circuit that performs processing on the satellite signal acquired by the second interface 1112. The baseband circuit performs, for example, filter processing, clock conversion processing, and Doppler removal processing on the satellite signal and writes a processing result in the storing section 113. It is assumed that the baseband circuit is realized by hardware. However, the baseband circuit is not prevented from being realized by software. The baseband circuit is not prevented from being omitted depending on a system of the GNSS.

The second processor 120 is electrically connected to the first processor 110 and acquires processing results of sensor signals (a biological signal, a body motion signal, and an environment signal) by the first processor 110. The second processor 120 can be realized by various processors such as a CPU, a DSP, and an ASIC.

Note that a not-shown interface may be provided between the first processor 110 and the second processor 120. The interface can be realized by an I2C, an SPI, a UART (Universal Asynchronous Receiver Transmitter), and the like.

The second processor 120 controls at least one of the display section 140 and the communication section 150 of the biological information measuring device 100. The display section 140 is a section for displaying various display screens and can be realized by, for example, a liquid crystal display and an organic EL display. The communication section 150 performs communication with an external device different from the biological information measuring device 100 via a network. The network can be realized by a WAN (Wide Area Network), a LAN (Local Area Network), a Bluetooth (registered trademark), an NFC (Near Field Radio Communication), and the like and may be either a wired network or a wireless network.

The biological sensor 131 can be realized by a pulse sensor, an arterial blood oxygen saturation sensor, a temperature (body temperature) sensor, and the like. The body motion sensor 132 can be realized by an acceleration sensor, a gyro sensor, an atmospheric pressure sensor, a terrestrial magnetism sensor, and the like. The environment sensor 133 can be realized by a temperature (environmental temperature) sensor, an illuminance sensor, a humidity sensor, an ultraviolet ray sensor, and the like.

The biological sensor 131, the body motion sensor 132, and the environment sensor 133 may be any one of the sensors described above or may be a combination of a plurality of the sensors. In the biological information measuring device 100, the body motion sensor 132 and the environment sensor 133 may be omitted.

In a method in this embodiment, acquisition of information from a sensor group (the biological sensor 131, the body motion sensor 132, and the environment sensor 133) and processing of the information can be performed by the first processor 110 and do not need to be performed through the second processor 120 (a host CPU). Therefore, it is possible to complete control of a GNSS circuit block and the sections of the first processor 110 on the inside of the first processor 110 (control by the host CPU is not essential).

From the viewpoint of the second processor 120, by providing the first processor 110 between the second processor 120 and the sensor group, desired information can be acquired in an appropriate form. For example, the second processor 120 can acquire, in a desired data format, desired information among physical quantities such as acceleration, angular velocity, a position (latitude and longitude), altitude, and atmospheric pressure. The second processor 120 may acquire not only simple physical quantities but also information calculated on the basis of the physical quantities. For example, the second processor 120 may acquire information representing an action determination result concerning what kind of action (exercise) a user is performing or may acquire information such as a pitch, a stride, and an air-stay time in running. Concerning the biological information, the second processor 120 is capable of acquiring not only raw data (e.g., pulse waveform information) but also information (a pulse rate, a pulse interval, and autonomic information) and the like calculated from the raw data.

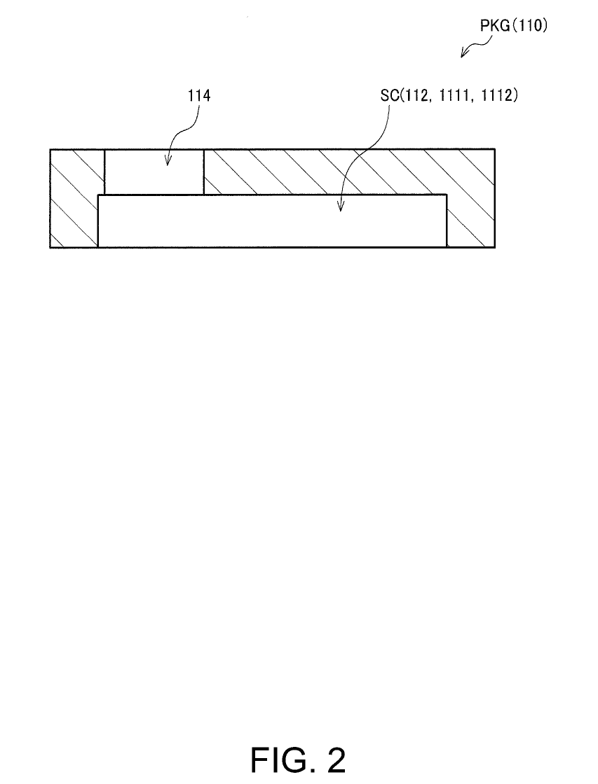

FIG. 2 is a configuration example of a package PKG of the first processor 110 according to this embodiment. As shown in FIG. 2, the processing section 112, the first interface 1111, and the second interface 1112 are formed on a semiconductor chip SC of one chip. Further, when the first processor 110 includes the nonvolatile memory 114, the nonvolatile memory 114 is stacked (disposed) on the semiconductor chip SC. In this case, the nonvolatile memory 114 is provided to be laminated on the semiconductor chip SC and is electrically connected by wire bonding. “Laminated” represents disposition in an overlapping position in the thickness direction of the package PKG (the up-down direction in FIG. 2). The package PKG is formed by sealing the semiconductor chip SC and the nonvolatile memory 114 (and a wire and a pad region) with resin or the like. In this embodiment, the nonvolatile memory 114 is not essential. The first processor 110 may be realized by the semiconductor chip SC of one chip.

Consequently, at least the processing section 112, the first interface 1111, and the second interface 1112 of the first processor 110 can be formed by one chip. However, desirably, all of the components excluding the nonvolatile memory 114 in the first processor 110 are formed on the semiconductor chip SC of one chip. Further, when the nonvolatile memory 114 is included in the first processor 110, the first processor 110 including the nonvolatile memory 114 can be configured by one package (the package PKG). The biological information measuring device 100 is configured by electrically connecting the package shown in FIG. 2, the second processor 120 (the host CPU) provided on a main substrate, the biological sensor 131 provided on a sensor substrate, and the like. However, various modified implementations are possible concerning a specific configuration of the biological information measuring device 100 to, for example, provide the second processor 120 and the biological sensor 131 on the same substrate.

Therefore, switching of operation clocks of the sections of the first processor 110 and fine power supply control of the storing section 113 (the memory) are possible. It is possible to construct an inexpensive and low-power consumption system. As explained above, the first processor 110 includes the processing section 112 (a sub-CPU). Therefore, processing for a sensor signal and control by the second processor 120 (the processing section and the host CPU) of the biological information measuring device 100 in the control of the sections are not essential.

In this embodiment, it is possible to acquire position information and the like of the user by processing the satellite signal with the processing section 112. However, in some case, the position information itself is unnecessary or it is sufficient to acquire the position information at a low frequency as in a clock display mode explained below. In that respect, in the first processor 110 in this embodiment, power supply control (ON/OFF control) of the GNSS circuit block (the second interface 1112 and the baseband circuit) is also possible. Therefore, for example, a further reduction in power consumption is also possible.

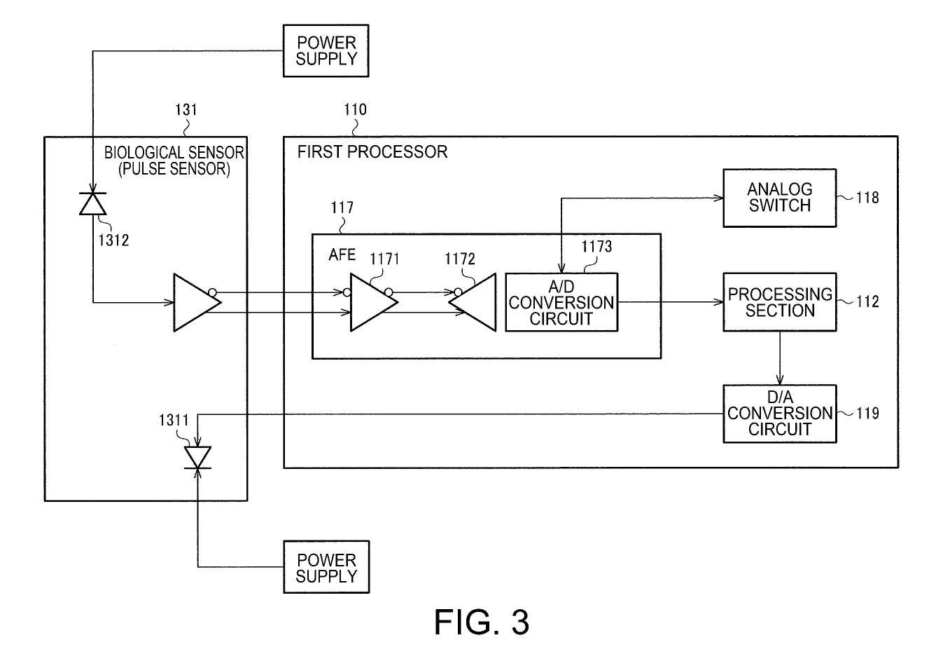

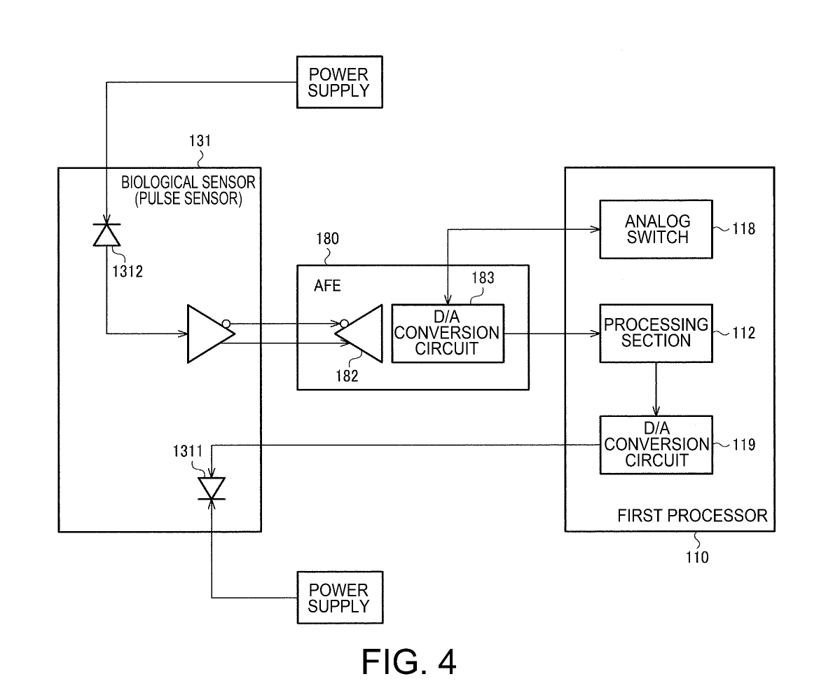

FIGS. 3 and 4 are diagrams for explaining connection examples of the biological sensor 131 and the first processor 110. An example in which a photoelectric pulse sensor is used as the biological sensor 131 is explained with reference to FIGS. 3 and 4.

The photoelectric pulse sensor includes a light emitting section 1311 (e.g., an LED (Light Emitting Diode)) and a light receiving section 1312 (e.g., a PD (Photodiode)). The light emitting section 1311 irradiates light in a wavelength band easily absorbed by blood (in a narrow sense, hemoglobin included in the blood). If a blood flow rate is high and an amount of the hemoglobin is large, an absorption amount of light is large and the intensity of reflected light is low. Conversely, if the blood flow rate is low and the amount of the hemoglobin is small, the absorption amount of the light is small and the intensity of the reflected light is high. In this case, since fluctuation (an AC component) of a signal output from the light receiving section 1312 represents fluctuation in the blood flow rate, an output signal of the light receiving section 1312 of the pulse sensor is a signal having a correlation with a pulse. That is, in the first processor 110, it is possible to calculate pulse wave information such as a pulse rate, a pulse interval, fluctuation in the pulse rate and the pulse interval, and the like.

However, since the output of the light receiving section 1312 is an analog signal (in a narrow sense, an analog voltage), an AFE (Analog Front End) that performs signal adjustment and A/D conversion is necessary between the light receiving section 1312 and the processing section 112 (in particular, the CPU or the like that performs digital processing). The first processor 110 in this embodiment may include the AFE or may use an external AFE.

FIG. 3 is a connection example of the pulse sensor and the first processor 110 that includes an AFE 117. As explained above, the pulse sensor includes the light emitting section 1311 and the light receiving section 1312. The signal of the light receiving section 1312 is output to the processing section 112 via the AFE 117. In this case, the first interface 1111 may be realized as the AFE 117. The AFE 117 includes an amplifier circuit 1171, a filter circuit 1172, and an A/D conversion circuit 1173.

Note that the first processor 110 may include an analog switch 118. The analog switch 118 is a switch for configuring a sample hold circuit in the A/D conversion circuit 1173. However, the A/D conversion circuit 1173 sometimes includes the sample hold circuit. In that case, the analog switch 118 of the first processor 110 can be omitted.

The light emitting section 1311 of the pulse sensor is an element that irradiates light having intensity corresponding to a supplied current value. Therefore, in order to control light emission timing and light emission intensity of the light emitting section 1311, the first processor 110 desirably outputs an analog signal to the light emitting section 1311 of the pulse sensor. Specifically, the first processor 110 includes a D/A conversion circuit 119. The D/A conversion circuit 119 D/A-converts digital data set by the processing section 112 and outputs an analog signal to the light emitting section 1311.

FIG. 4 is a connection example of the pulse sensor and the first processor 110 that uses an external AFE 180. The AFE 180 includes a filter circuit 182 and an A/D conversion circuit 183. A signal of the light emitting section 1311 is input to the AFE 180. Digital data from the A/D conversion circuit 183 is input to the processing section 112 via the first interface 1111. The first interface 1111 in this case can be realized by a serial interface or the like such as an I2C or an SPI.

Note that, as shown in FIGS. 3 and 4, the first processor 110 may include the analog switch 118 and the D/A conversion circuit 119 irrespective of presence or absence of the AFE. Since the analog switch 118 and the D/A conversion circuit 119 are relatively large, by incorporating the analog switch 118 and the D/C conversion circuit 119 into the first processor 110 (the semiconductor chip SC), it is possible to reduce the size and the number of components of the biological information measuring device 100.

Note that the connection examples of the pulse sensor and the first processor 110 are explained above. However, connection by various forms is also possible concerning the other sensors included in the sensor group.

The method in this embodiment can be applied to a wearable device 200 including the biological information measuring device 100. Since the wearable device 200 is worn on the user and used, it is easy to detect body motion information and biological information. That is, the biological sensor 131 and the body motion sensor 132 are often provided in the wearable device 200. Importance of processing of sensor information is large. In order to reduce a user load due to the device wearing, the wearable device 200 is desirably small in size and weight. Therefore, a battery capacity is often small. A reduction in power consumption is also important. In that respect, since the biological information measuring device 100 in this embodiment can realize processing of sensor information with low power consumption, affinity with the wearable device 200 is considered to be extremely high.

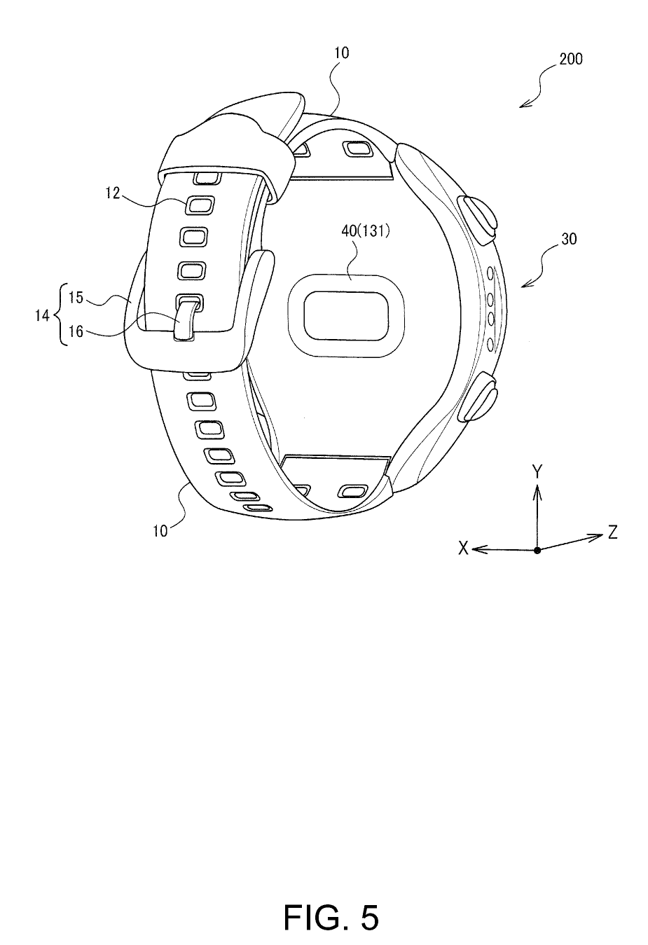

FIG. 5 is an example of an exterior view of the wearable device 200. As shown in FIG. 5, the wearable device 200 includes a case section 30 and a band section 10 for fixing the case section 30 to the body (in a narrow sense, a wrist) of the user. Fitting holes 12 and a buckle 14 are provided in the band section 10. The buckle 14 is configured from a buckle frame 15 and a locking section (a protrusion bar) 16.

FIG. 5 is a perspective view of the wearable device 200, which is in a state in which the band section 10 is fixed using the fitting hole 12 and the locking section 16, viewed from the direction on the band section 10 side (a surface side, which is a subject side in a worn state, of the surface of the case section 30). In the wearable device 200 shown in FIG. 5, a plurality of the fitting holes 12 are provided in the band section 10. The wearable device 200 is worn on the user by inserting the locking section 16 of the buckle 14 into any one of the plurality of fitting holes 12. The plurality of fitting holes 12 are provided along the longitudinal direction of the band section 10 as shown in FIG. 5.

A sensor section 40 is provided in the case section 30 of the wearable device 200. The sensor section 40 may be the biological sensor 131 and may include the body motion sensor 132 and the environment sensor 133. In FIG. 5, assuming that the sensor section 40 is the biological sensor 131 (in particular, a photoelectric pulse sensor), an example is shown in which the sensor section 40 is provided on a surface of the case section 30 on the subject side during wearing of the wearable device 200. However, a position where the sensor is provided is not limited to the position shown in FIG. 5. For example, the body motion sensor 132 may be provided on the inside of the case section 30 (in particular, on a sensor substrate included in the case section 30).

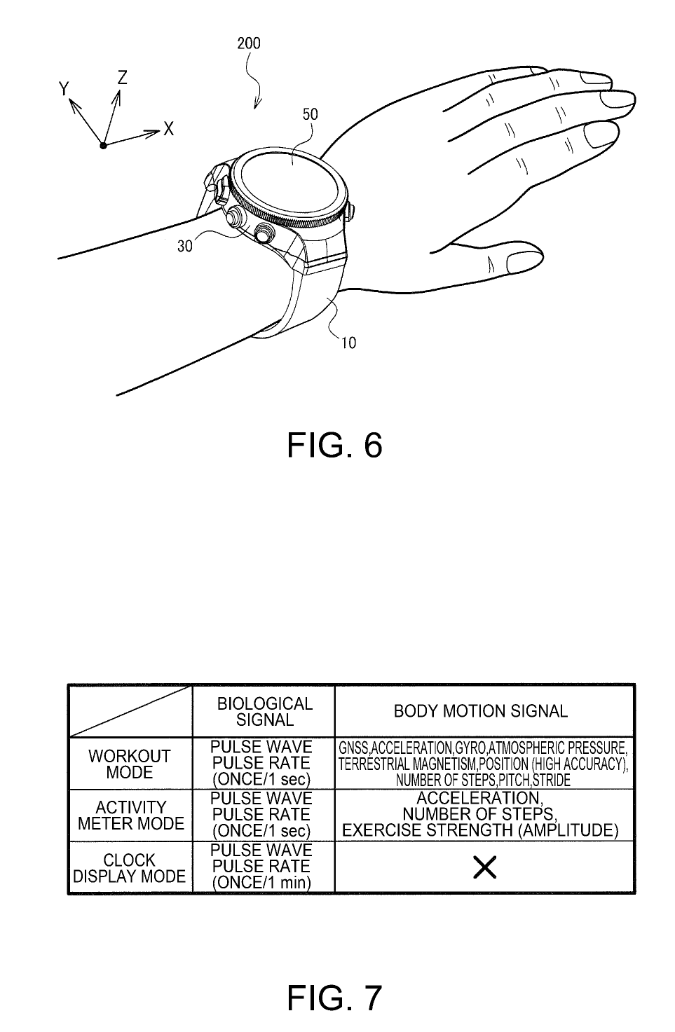

FIG. 6 is a view of the wearable device 200 worn by the user viewed from a side on which a display section 50 (corresponding to the display section 140 shown in FIG. 1) is provided. As it is seen from FIG. 6, the wearable device 200 in this embodiment includes the display section 50 in a position equivalent to a dial of a normal wristwatch or a position where numbers and icons can be visually recognized. In the worn state of the wearable device 200, the surface of the case section 30 on the side shown in FIG. 5 adheres to the subject. The display section 50 is in a position where visual recognition by the user is easy.

Note that, in FIGS. 5 and 6, a coordinate system is set on the basis of the case section 30 of the wearable device 200. A direction crossing a display surface of the display section 50, that is, a direction from a rear surface to a front surface when the display surface side of the display section 50 is represented as the front surface is set as a Z-axis positive direction. Alternatively, a direction from the sensor section 40 (in a narrow sense, the pulse sensor shown in FIG. 5) to the display section 50 or a direction away from the case section 30 in the normal direction of the display surface of the display section 50 may be defined as the Z-axis positive direction. In a state in which the wearable device 200 is worn on the subject, the Z-axis positive direction is equivalent to a direction from the subject to the case section 30. Two axes orthogonal to the Z axis are set as X and Y axes. In particular, a direction in which the band section 10 is attached to the case section 30 is set as the Y axis.

As the wearable device 200, an example of a device retained on the arm (the wrist) of the user by the band section 10 is explained with reference to FIGS. 5 and 6. However, a shape and a wearing part of the wearable device 200 are not limited to the shape and the wearing part shown in FIGS. 5 and 6. For example, the wearable device 200 may be a device worn on other parts of the user such as an ankle by the band section 10 or may be an HMD (Head Mounted Display) or the like.

### 2. Operation Modes and Operation Programs

The biological information measuring device 100 (the first processor 110) in this embodiment has a plurality of operation modes and is capable of operating in any one of the plurality of operation modes. The plurality of operation modes may include at least two of a clock display mode, an activity meter mode, and a workout mode.

The workout mode is an operation mode corresponding to a case in which the user is performing a workout. The workout represents movement and training of the body and corresponds to exercise such as sports. In the workout mode, the user strenuously moves the body and, therefore, states of biological activities such as a pulse rate greatly change. In the workout mode, it is also important to calculate not only simple strength of a body motion but also detailed information such as a direction and a frequency of the body motion like calculation of a pitch and a stride in running.

Therefore, in the workout mode, the first processor 110 desirably performs at least one of the following (1) to (3). Note that, in the following explanation, “relatively” represents comparison with other operation modes.

(1) Relatively increase a calculation frequency based on sensor signals output from the sensors

(2) Relatively increase types of sensors in use

(3) Relatively increase a load (accuracy) of calculation based on the sensor signals

FIG. 7 is a diagram showing an example of the operation modes and processing contents of the first processor 110 corresponding to the operation modes. More specifically, FIG. 7 shows processing contents in the case in which the processing section 112 of the first processor 110 operates according to operation programs (applications) corresponding to the operation modes.

For example, in the workout mode, the first processor 110 executes, once in one second, arithmetic processing of a pulse rate based on a sensor signal output from the pulse sensor. In the example shown in FIG. 7, this rate is high compared with the clock display mode. (1) described above is realized. Consequently, it is possible to analyze changes in the biological activities.

The first processor 110 in the workout mode sets all of sensor signals transmitted from the GNSS and output from the pulse sensor, the acceleration sensor, the gyro sensor, the atmospheric pressure sensor, and the terrestrial magnetism sensor as processing targets. In the example shown in FIG. 7, processing target sensor types are large compared with the other modes such as the activity meter mode. (2) described above is realized. Consequently, it is possible to include various sensing results in the processing targets.

In the workout mode, the first processor 110 performs detailed operation state determination. For example, as processing based on the sensor signals transmitted from the GNSS and output from the acceleration sensor and the gyro sensor, the first processor 110 performs processing for calculating a pitch, a stride, and the like. Specifically, the first processor 110 calculates position information of the user according to inertial navigation in which information of the GNSS and the acceleration sensor and the gyro sensor are used. Further, the first processor 110 performs processing for extracting a vertical component from acceleration information of the acceleration sensor and processing for calculating the number of steps from a peak interval and a frequency of the vertical component. The first processor 110 performs processing for calculating a pitch and a stride from a distance (displacement of a position), the number of steps, a time, and the like. In the example shown in FIG. 7, there are more contents of the arithmetic processing compared with the activity meter mode and the like. (3) described above is realized. Consequently, it is possible to analyze not only presence or absence of exercise and the magnitude of strength of the exercise but also a more detailed exercise state.

Note that it goes without saying that the first processor 110 in the workout mode may perform processing relatively having a high load (accuracy) targeting the biological signal. For example, it is possible to add processing for estimating an autonomic nerve state and estimating an arrhythmia risk by calculating not only a pulse rate but also fluctuation in the pulse rate.

The workout mode is not prevented from being further divided into a plurality of modes. For example, a normal workout mode corresponding to a workout with a relatively small load among workouts and a high-load workout mode corresponding to a workout with a relatively large load among the workouts may be provided.

The normal workout mode corresponds to exercise such as walking. The high-load workout mode corresponds to, for example, interval training including a short-distance dash and weight training. In this case, in the high-load workout mode, the first processor 110 desirably sets at least one of a calculation frequency, sensor types, and a calculation load (accuracy) high (more) compared with the normal workout mode.

The activity meter mode is a mode for measuring an index value (an activity amount) of an activity of the user. The activity is different from the workout and represents an activity in daily life such as housework, work, or study. In the daily life, it is assumed that a body motion is not as large as the body motion in the workout and a state of a biological activity does not suddenly change. Therefore, as the activity amount, for example, a total value of the numbers of steps in one day only has to be able to be displayed. Necessity of detailed information such as changes in a pitch and a stride is low. Accuracy of position information is unnecessary compared with the workout mode.

Therefore, the first processor 110 in the activity meter mode excludes the GNSS from the processing targets as shown in FIG. 7. In the activity meter mode, the first processor 110 also excludes the body motion sensors (the gyro sensor, the atmospheric pressure sensor, the terrestrial magnetism sensor, and the like) other than the acceleration sensor from the processing targets. Further, the first processor 110 omits calculation of the inertial navigation and calculation of a pitch and a stride. For example, the first processor 110 in the activity meter mode calculates the magnitude of an acceleration value as information representing exercise strength and performs calculation of only the number of steps.

In FIG. 7, an example is explained in which the first processor 110 in the activity meter mode performs calculation of a pulse rate at the same frequency (e.g., once in one second) as the frequency in the workout mode. However, the first processor 110 is not prevented from reducing a calculation rate and providing a difference in types of biological information to be calculated.

A sleep mode may be provided separately from the activity meter mode. The sleep mode is an operation mode corresponding to a case in which it is determined that the user is in a sleeping state. In the sleeping state, a body motion is still smaller compared with the state in which the user performs the daily life such as housework. Necessity of determining a detailed exercise state is low. Therefore, in the sleep mode, for example, calculation of a pulse rate is reduced to a low frequency of once in one minute (or once in five minutes or once in ten minutes). Concerning an acceleration signal output from the acceleration sensor, a calculation rate may be reduced in the same manner. The acceleration signal itself may be excluded from the processing targets.

The clock display mode is a mode for performing time display and is a mode for minimizing measurement of biological information and body motion information of the user. Therefore, the first processor 110 in the clock display mode performs processing for acquiring time information from a clocking section and outputting the time information. The clocking section is, for example, a real-time clock (RTC) provided in the biological information measuring device 100 and may be provided either on the inside or the outside of the first processor 110.

By providing the operation modes as explained above, it is possible to perform appropriate processing of sensor information corresponding to an activity state of the user. For example, in the workout mode, it is possible to detect detailed information of the user. In the activity meter mode and the clock display mode, it is possible to reduce a processing load and power consumption by selecting information.

Switching of the operation mode may be performed on the basis of a biological signal. Specifically, the processing section 112 performs processing for switching the operation mode on the basis of the biological signal. Alternatively, body motion information may be used for the switching of the operation mode. The processing section 112 performs, on the basis of at least one of the biological signal and the body motion signal, the processing for switching the operation mode.

Consequently, it is possible to perform operation mode switching corresponding to a state of a biological activity and a state of a body motion of the user detected by the sensors. Therefore, the biological information measuring device 100 is capable of performing processing matched with a state of the user.

For example, when biological information is used, the operation mode may be switched according to the magnitude of a pulse rate. In general, in a workout state, the pulse rate is large compared with a state in which a daily activity is performed. Various forms of use of the clock display mode are conceivable. For example, it is conceivable to set the operation mode to the clock display mode in a state in which an activity of the user is sufficiently suppressed (a rest time or the like).

Therefore, when given thresholds Th1 and Th2 (>Th1) are set, if a value HR of a pulse rate is HR<Th1, the processing section 112 determines that the user is in a rest state and sets the operation mode to the clock display mode. Similarly, if Th1≤HR<Th2, the processing section 112 determines that the user is in a daily life state and sets the operation mode to the activity meter mode. If Th2≤HR, the processing section 112 determines that the user is in a workout state and sets the operation mode to the workout mode.

When body motion information is used, similarly, the magnitude of a body motion (exercise strength) is assumed to be in a relation of the rest state<the daily activity state<the workout state. Therefore, the processing section 112 only has to determine according to comparison of the magnitude of the exercise intensity and a given threshold which of the plurality of operation modes is executed.

The processing section 112 may combine the biological information and the body motion information and determine which of the plurality of operation modes is executed. Further, for example, matching processing of feature values of the biological information and the body motion information and given reference feature values may be executed rather than the simple comparison with the thresholds explained above. Besides, various methods are known concerning state determination (action determination) for the user. In this embodiment, it is possible to widely apply the methods.

Note that the first processor 110 (the processing section 112) has to be able to acquire information used for the selection of the operation mode even in the clock display mode. For example, if the pulse rate is used for the selection of the operation mode as explained above, the first processor 110 needs to perform the arithmetic processing of the pulse rate even in the clock display mode. In the example shown in FIG. 7, the first processor 110 in the clock display mode performs the calculation of the pulse rate at a low frequency of once in one minute.

### 3. Software Configuration Example

The plurality of operation modes are respectively realized by operation programs (applications) corresponding to the operation modes. Specifically, the processing section 112 of the first processor 110 operates according to an operation program, whereby processing in the operation mode corresponding to the operation program is executed. In this case, the first processor 110 in this embodiment may not include the nonvolatile memory 114. The plurality of operation programs may be stored in the ROM 1132. In this case, since it is difficult to add and change the operation programs, it is necessary to create operation programs assumed as necessary in advance.

The first processor 110 not including the nonvolatile memory 114 can be configured by, for example, one chip like the semiconductor chip SC in FIG. 2. Therefore, it is possible to realize the first processor 110 inexpensively and in a small size. However, when a plurality of devices, sensors in use and kinds of processing to be executed (operation modes) of which are different, are prepared as the biological information measuring device 100, it is necessary to use the first processor 110 corresponding to the devices.

Alternatively, the first processor 110 may include the nonvolatile memory 114 that stores a plurality of operation programs corresponding to the respective plurality of operation modes. The plurality of operation programs are retained in the nonvolatile memory 114 and can be added and changed. Therefore, it is possible to flexibly set what kind of information is output in what kind of form. That is, when the biological information measuring device 100 is configured, it is possible to flexibly set a configuration of the sensor group and a type and a form of information acquired by the second processor 120 (the host CPU) from the first processor 110. In other words, it is possible to easily realize a variety of biological information measuring devices 100 using the first processor 110 including the nonvolatile memory 114. It is also possible to, for example, reduce cost and power consumption and reduce the number of components of the biological information measuring devices 100.

A configuration example in a software level of the biological information measuring device 100 (the first processor 110) is explained on the premise of the hardware configuration shown in FIG. 1. A first embodiment, a second embodiment, and modifications are explained below.

### 3.1 First Embodiment

As explained above, as hardware, the sensor group (the biological sensor 131, the body motion sensor 132, etc.), the storing section 113 (the RAM 1131 and the ROM 1132) of the first processor 110, the nonvolatile memory 114, and the processing section 112 provided in the biological information measuring device 100 or the like are provided.

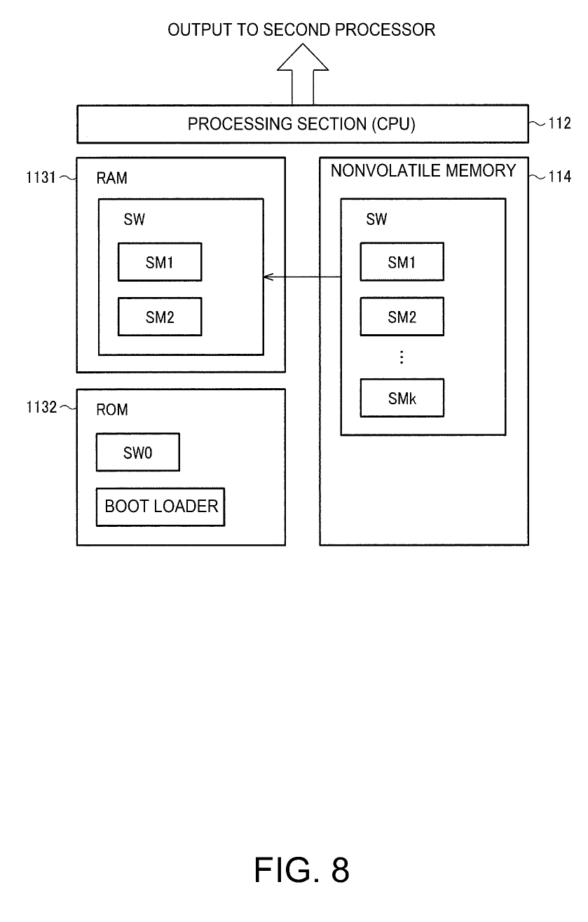

FIG. 8 is a software configuration example in the first embodiment. In the nonvolatile memory 114, software SW including an OS (Operating System) and sub-modules SM1 to SMk capable operating on the software SW are stored, where k is an integer equal to or larger than 2. The software SW and a selected sub-module among the sub-modules SM1 to SMk are loaded to the RAM 1131.

The sub-modules SM1 to SMk include modules that perform processing based on sensor signals. For example, a module that acquires information output from the pulse sensor and calculates a pulse rate and a module that acquires information output from the acceleration sensor and calculates action information of the user are conceivable. Note that the calculation module for action information is not prevented from being divided into a number-of-steps calculation module, a pitch calculation module, a stride calculation module, and the like. A module that performs general-purpose processing, in which sensor information is not set as a processing target, is not prevented from being included in a part of the sub-modules SM1 to SMk.

The respective plurality of operation programs in this embodiment are realized by one or a combination of a plurality of the sub-modules SM1 to SMk. For example, when the biological-information measuring device 100 (the first processor 110) operates in a first operation mode, a first operation program corresponding to the first operation mode is loaded to the RAM 1131. The processing section 112 performs operation in the first operation mode according to the first operation program. More specifically, one or a plurality of sub-modules corresponding to processing set as execution targets in the first operation mode among the sub-modules SM1 to SMk are loaded to the RAM 1131.

Concerning the other operation programs, similarly, sub-modules corresponding to the operation programs are loaded from the nonvolatile memory 114 to the RAM 1131 and the processing section 112 operates using the loaded sub-modules.

Switching of the sub-module can be realized by control for adding (deleting) a sub-module loaded to the RAM 1131 every time processing content increases (decreases) or control for replacing a loaded sub-module with another sub-module.

A change of the operation mode, that is, switching of the operation program is performed by software SW (OS) stored in the nonvolatile memory 114, loaded to the RAM 1131, and executed. Specifically, the processing section 112 operates according to the software SW to perform processing for switching the operation program.

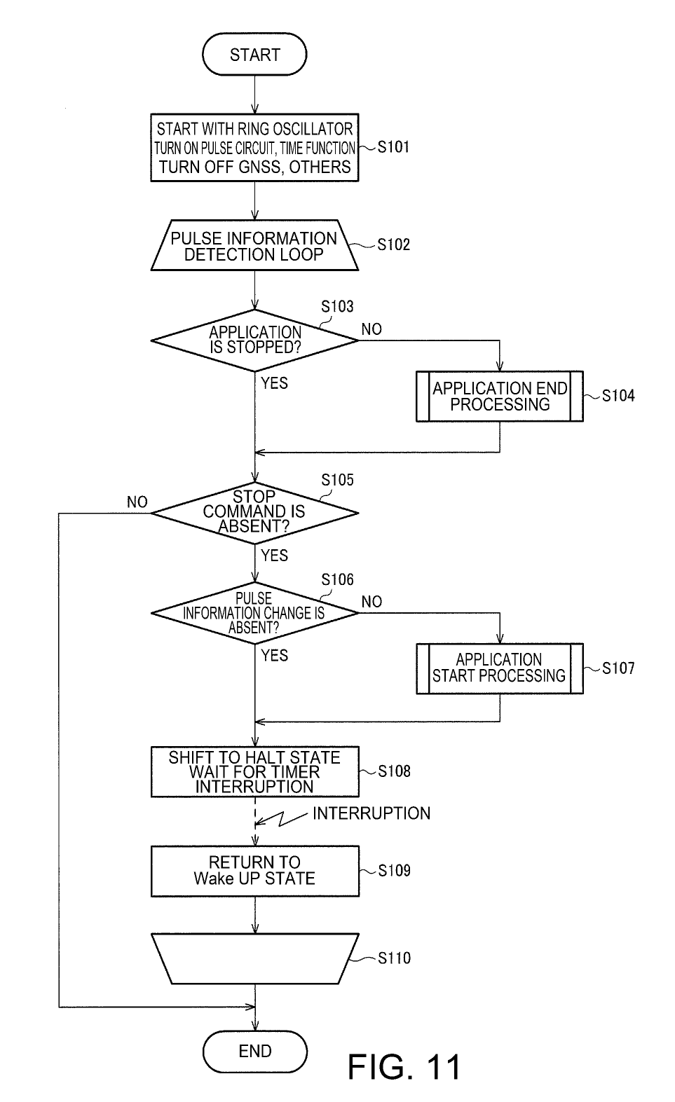

As explained above, the operation program loaded to the RAM 1131 may be switched on the basis of at least one of the biological signal and the body motion signal. For example, information based on the biological signal is acquired (calculated) by the operation program (the sub-module) and the software SW periodically checks the acquired information. When determining that the information satisfies a given switching condition, the software SW performs processing for switching the operation program (the sub-module) loaded to the RAM 1131. A specific flow of operation is explained below with reference to flowcharts of FIGS. 11 to 13.

In the above explanation, the plurality of operation programs are stored in the nonvolatile memory 114. However, a given operation program SW0 may be stored in the ROM 1132. It is optional which operation modes the operation program stored in the ROM 1132 corresponds to. For example, an operation program corresponding to the workout mode may be stored in the nonvolatile memory 114 as explained above and an operation program corresponding to the activity meter mode and the clock display mode may be stored in the ROM 1132 as the software SW0. Alternatively, an operation program (a boot loader) corresponding to a start mode executed at a start time of the biological information measuring device 100 may be stored in the ROM 1132. The operation program stored in the ROM 1132 is not limited to one. A plurality of operation programs corresponding to the plurality of operation modes may be stored.

The software SW for switching the operation mode (the operation program) selects either one of the operation program loaded to the RAM 1131 from the nonvolatile memory 114 and the operation program SW0 stored in the ROM 1132. That is, the processing section 112 operates according to either one of the operation program stored in the ROM 1132 and the operation program loaded to the RAM 1131.

Consequently, in addition to the plurality of operation programs stored in the nonvolatile memory 114, the operation program stored in the ROM 1132 can also be set as a selection target. For example, an operation mode with high versatility and an operation mode with low necessity of expansion can be executed by being created in the ROM 1132 in advance. The operation mode with low necessity of expansion represents an operation mode in which rewriting by the user who uses the biological information measuring device 100 is not assumed like, for example, the clock display mode.

### 3.2 Second Embodiment

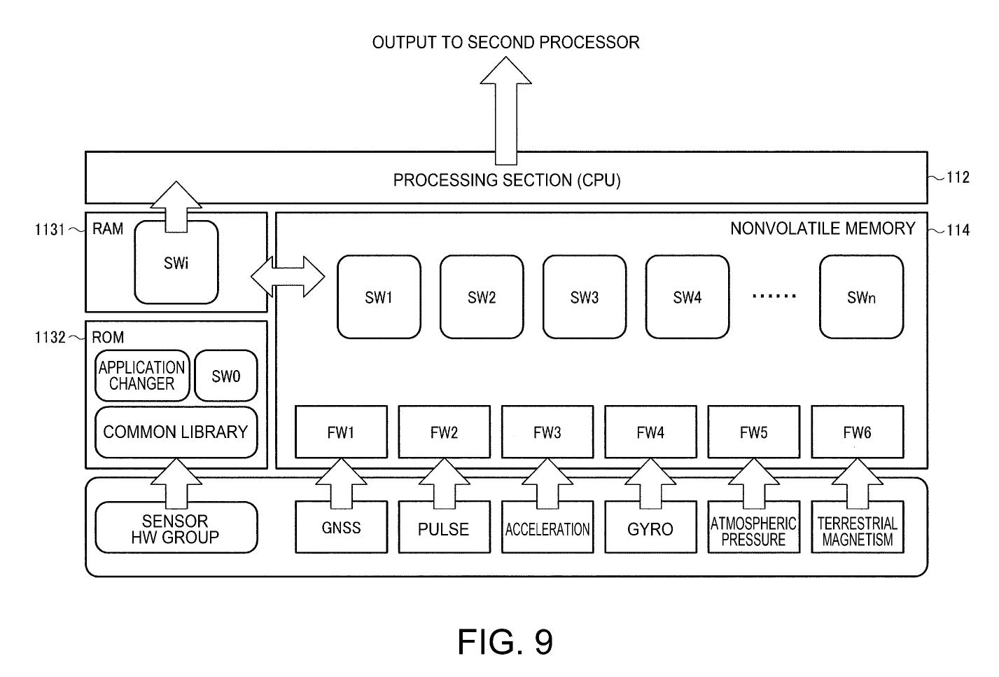

FIG. 9 is a software configuration example in a second embodiment. In the nonvolatile memory 114, kinds of firmware FW1 to FWm and operation programs SW1 to SWn are stored. In the ROM 1132, an application changer and a common library are stored. A selected operation program SWi (i is an integer equal to or larger than 1 and equal to or smaller than n) among the plurality of operation programs is loaded to the RAM 1131. The processing section 112 operates according to the operation program SWi loaded to the RAM 1131.

The kinds of firmware FW1 to FWm acquire sensor signals transmitted from the GNSS and the sensor group via the first to fourth interfaces 1111 to 1114 and output the sensor signals in a data format usable in higher-order software. Specifically, the kinds of firmware FW1 to FWm are provided to correspond to the sensors. In the example shown in FIG. 9, the firmware FW1 is firmware corresponding to GNSS. The firmware FW1 acquires and outputs a GNSS signal (a signal received by the antenna and output via conversion processing by the RF circuit and the baseband circuit). Similarly, the kinds of firmware FW2 to FW6 respectively acquire and output a pulse signal transmitted from the pulse sensor, an acceleration signal transmitted from the acceleration sensor, an angular velocity signal transmitted from the gyro sensor, an atmospheric pressure signal transmitted from the atmospheric pressure sensor, and a terrestrial magnetism signal transmitted from the terrestrial magnetism sensor. In this case, the m is an integer of magnitude corresponding to the number of types of acquisition target sensor signals. In the example shown in FIG. 9, m=6.

The kinds of firmware FW1 to FWm perform processing for acquiring the sensor signals, for example, at a frequency corresponding to an output rate of the sensors and storing (outputting) the sensor signals in association with acquisition time information. The acquisition time information may be absolute time such as UTC (Coordinated Universal Time) or JST (Japan Standard Time) or may be information such as a timestamp. The kinds of firmware FW1 to FWm are not prevented from performing processing such as data conversion.

The operation programs SW1 to SWn are software for operating (operating the processing section 112) on the basis of the sensor signals acquired by the kinds of firmware FW1 to FWn. The n is an integer having a magnitude corresponding to the number of operation modes. The example of the operation modes and the example of the processing in the operation modes are explained above. Therefore, detailed explanation of the operation modes and the processing in the operation modes is omitted. For example, the operation program SW1 is an operation program corresponding to the workout mode. The operation program SW2 is an operation program corresponding to the activity meter mode. The operation program SW3 is an operation program corresponding to the clock display mode.

In the second embodiment, a change of the operation mode, that is, switching of the operation program is performed by the application changer stored in the ROM 1132. Specifically, the processing section 112 operates according to the application changer to perform processing for switching the operation program. That is, the switching of the operation program may be executed by software in a RAM region as in the first embodiment or may be executed by software in a ROM region as in the second embodiment.

Note that, as it is seen from FIG. 7 as well, there is processing executed in common in a plurality of operation programs. In the example shown in FIG. 7, the calculation of a pulse rate based on information output from the pulse sensor (the output of the FW2) is performed in all of the workout mode, the activity meter mode, and the clock display mode. Alternatively, the calculation of the number of steps based on information output from the acceleration sensor (the output of the FW3) is executed in common in the workout mode and the activity meter mode.

In this way, processing with high versatility and processing with high importance may be managed as a library (a software library). In the example shown in FIG. 9, a common library is stored in the ROM 1132. The common library is a library for sharing the processing with high versatility and high importance explained above in the plurality of operation programs. The common library may be, for example, an API (Application Programming Interface). The common library (the API) can be used (called) in all of the plurality of operation programs (SW1 to SWn).

In FIG. 9, an example is shown in which a hierarchical structure of two layers of the firmware (FW1 to FWm) and the operation programs (SW1 to SWn) is used. However, not only this, but, for example, software that is an intermediate layer between the firmware and the operation program may be used. In the software, for example, processing performed by integrating information output from the plurality of sensors, that is, processing with high versatility is executed. Consequently, since the processing with high versatility can be left to the software in the intermediate layer, it is possible to facilitate packaging of the operation programs. The hierarchical structure is not limited to two layers or three layers and can be expanded to four or more layers.

### 3.3 Modifications

Various modified implementations of the software configuration in this embodiment are possible. For example, the first embodiment and the second embodiment may be combined. Specifically, the common library explained in the second embodiment may be stored in the ROM 1132 in the first embodiment. Besides, a modified implementation is possible in which the configuration of one embodiment is incorporated in the other embodiment.

The method in this embodiment can be applied to a sensor information processing device including a processing section (a processor), an interface that acquires a biological signal output from the biological sensor 131, a nonvolatile memory (Flash) that stores the plurality of operation programs corresponding to the plurality of operation modes, and a storing section (a RAM (Random Access Memory)). The operation programs are computer programs operating on the basis of sensor information output from the sensor group. The operation program selected out of the plurality of operation programs is loaded to the storing section. The processing section operates according to the operation program loaded to the storing section.

The processing section corresponds to the processing section 112 shown in FIG. 1. The interface corresponds to the first interface 1111. The nonvolatile memory corresponds to the nonvolatile memory 114 shown in FIG. 1. The storing section corresponds to the RAM 1131. However, the sensor information processing device may acquire sensor signals (sensor information) output from the sensors other than the biological sensor 131 such as the body motion sensor 132 and the environment sensor 133. In that case, the interface corresponds to at least one of the first interface 1111, the third interface 1113, and the fourth interface 1114.

That is, the sensor information processing device corresponds to the first processor 110 including the nonvolatile memory 114 and capable of switching and executing the plurality of operation programs stored in the nonvolatile memory 114.

In the method in this embodiment, sensor information (a biological signal) output from the biological sensor is acquired via the interface. The processing section operates according to the operation program loaded to the storing section from the nonvolatile memory to execute processing based on the sensor information. Therefore, in the sensor information processing device in this embodiment, it is possible to perform acquisition of sensor information and appropriate processing. The operation programs stored in the nonvolatile memory can be added and changed. Therefore, it is also easy to add a new sensor to processing targets and perform different processing on the sensor information. In other words, in the method in this embodiment, it is possible to realize the sensor information processing device (and an electronic device including the sensor information processing device) having high extendibility.

### 4. Circuit Control Corresponding to an Operation Program

In the biological information measuring device 100 (the first processor 110), hardware control may be switched according to an operation program (an operation mode). In the following explanation, a clock signal, a power supply, and communication are respectively explained.

### 4.1 Clock Signal

As shown in FIG. 1, the biological information measuring device 100 (the first processor 110) in this embodiment includes the clock supply section 115 that receives an input of a plurality of clock signals and supplies a selected clock signal among the plurality of clock signals.

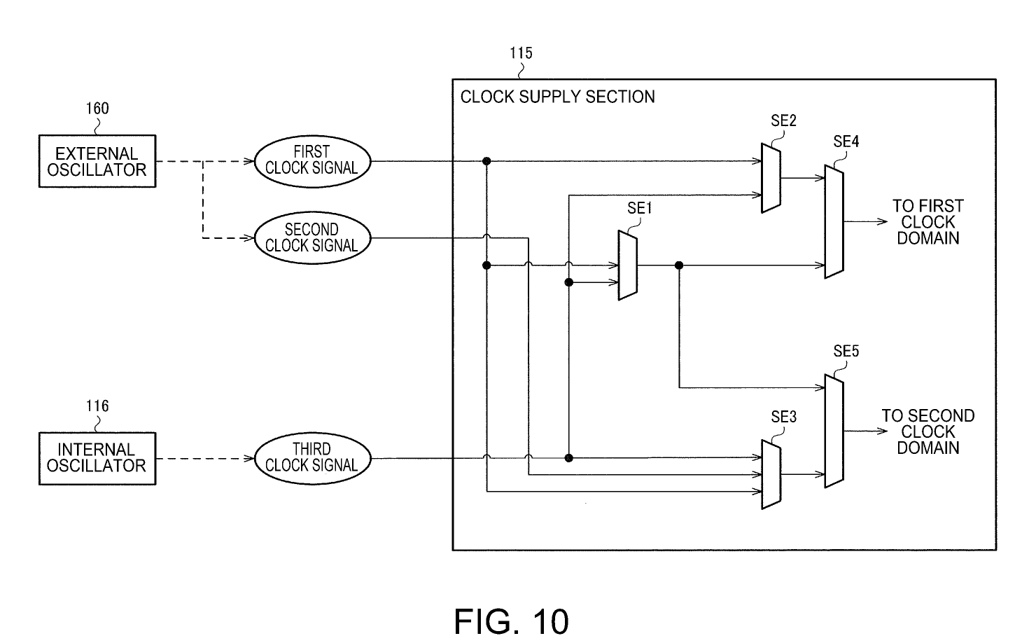

FIG. 10 is a configuration example of the clock supply section 115. A clock signal in this embodiment includes a signal output from the external oscillator 160 and a signal output from the internal oscillator 116. Specifically, as shown in FIG. 10, a first clock signal and a second clock signal based on the external oscillator 160 (the TCXO) and a third clock signal based on the internal oscillator 116 (the ring oscillator) are input to the clock supply section 115.

The first clock signal is a clock signal output from the external oscillator 160. The second clock signal is a signal obtained by multiplying the signal output from the external oscillator 160. That is, the clock signal may include the signal obtained by multiplying the signal output from the external oscillator 160. When the GNSS is used, a signal having an extremely high frequency such as 1575.42 MHz is input from the antenna. Therefore, in the biological information measuring device 100, by using the signal and the external oscillator 160 (the TCXO), it is possible to generate the second clock signal having a high frequency compared with the signal output from the external oscillator 160.

The third clock signal is a clock signal output from the internal oscillator 116 and is, for example, a signal having a frequency of the same degree as the frequency of the first clock.

The clock supply section 115 includes selectors SE1 to SE5. The first clock signal and the third clock signal are input to the selector SE1. The first clock signal and the third clock signal are input to the selector SE2. The first to third clock signals are input to the selector SE3. An output signal of the selector SE1 and an output signal of the selector SE2 are input to the selector SE4. The output signal of the selector SE1 and an output signal of the selector SE3 are input to the selector SE5.

A plurality of clock domains may be set in the biological information measuring device 100 (the first processor 110) in this embodiment. A clock signal, which is an output of the selector SE4, is supplied to a first clock domain. A clock signal, which is an output of the selector SE5, is supplied to a second clock domain. The first clock domain is, for example, a circuit block (a baseband circuit) corresponding to the GNSS. The second clock domain is a domain operable at a clock having a high frequency compared with the first clock domain. For example, the processing section 112 and the like are included in the second clock domain.

The clock supply section 115 in this embodiment may perform selection of a clock signal supplied at a start time and selection of a clock signal supplied at an operation time after the start. The start time means a period in which the first processor 110 (or the entire biological information measuring device 100) shifts from a stop state, a halt state, or a standby state to a normal operation state. The operation time represents a period after the start ends and the first processor 110 shifts to the normal operation state.

In the example shown in FIG. 10, the selector SE1 selects a clock signal supplied at the start time. The selector SE2 selects a clock signal supplied to the first clock domain at the operation time after the start. The selector SE3 selects a clock signal supplied to the second clock domain at the operation time after the start.

The selector SE4 selects an output signal of the selector SE1 at the start time and selects an output signal of the selector SE2 at the operation time after the start. The selector SE5 selects the output signal of the selector SE1 and selects an output signal of the selector SE3 at the operation time after the start. Consequently, it is possible to respectively select clock signals at the start time and the operation time. Further, it is also possible to execute the selection of clock signals on the respective plurality of clock domains.

At the start time, for example, acquisition of a sensor signal (a biological signal) is considered to be not performed either. Therefore, the selection of a clock signal in the selector SE1 only has to be performed on the basis of, for example, a clock selection signal transmitted from the outside.

The clock supply section 115 may select, on the basis of a biological signal, a clock signal to be supplied. Consequently, it is possible to supply an appropriate clock signal corresponding to a situation.

For example, when it is determined on the basis of the biological signal that the user is in the workout state, the operation program SW1 is loaded and executed in terms of software as explained above. In the processing in the operation program SW1, since information of the various sensors including the GNSS is necessary, the external oscillator 160 and a circuit that generates the first and second clock signals are considered to be also operating. Therefore, in this case, the clock supply section 115 only has to select and supply the first clock signal or the second clock signal rather than the third clock signal. When data processing at high speed is particularly necessary as in the high-load workout mode, the clock supply section 115 desirably selects and supplies the second clock signal having a high frequency rather than the first clock signal.

On the other hand, in the activity meter mode and the clock display mode, the GNSS is unnecessary. It is likely that a GNSS-based circuit is not operating as explained below. In that case, since the first and second clock signals are not input, the clock supply section 115 desirably selects and supplies the third clock signal.

The example in which any one of the first to third clock signals is supplied from the clock supply section 115 is explained above. However, other clock signals may be supplied from the clock supply section 115. For example, it is known that a clock signal used in the GNSS is different depending on a system (e.g., different in the GPS and the GLONASS). Therefore, the clock supply section 115 may supply a clock signal having a frequency different from the frequency of the first clock signal to the first clock domain.

A clock signal output from an external oscillator different from the TCXO and a clock signal, a clock source of which is a real-time clock, may be input to the clock supply section 115. The clock supply section 115 may supply the clock signals.

The sections of the biological information measuring device 100 may directly use a clock signal supplied from the clock supply section 115. However, not only this, but the sections of the biological information measuring device 100 may divide and use the clock signal. For example, in the clock display mode explained above, since it is sufficient to perform processing at low speed, the processing section 112, the storing section 113, and the like may operate according to a clock signal obtained by dividing the third clock signal into a half, a quarter, or the like. In this case, the processing section 112 and the like are capable of operating at multiple stages such as low speed (the divided clock signal), normal (the first or third clock signal), and high speed (the second clock signal).

### 4.2 Power Supply

The biological information measuring device 100 (the first processor 110) may control power supply to the sections according to an operation mode (an operation program). For example, processing for performing power supply control for the sections is defined in the operation program. The processing section 112 operates according to the operation program to perform, for example, output of a control signal. Consequently, it is possible to stop operation of unnecessary hardware in an execution target operation mode. Therefore, it is possible to reduce power consumption. Note that, in the following explanation, ON/OFF of a power supply is explained. However, a modified implementation for, for example, performing intermittent operation (reducing an operation frequency) is also possible.

First, the first processor 110 (the processing section 112) may perform power supply control for the storing section 113. The storing section 113 is the RAM 1131 in a narrow sense. The RAM 1131 includes a plurality of storage regions. Among the plurality of storage regions, a storage region, to which electric power is supplied, is switched according to a selected operation mode.

For example, the storing section 113 includes p (p is an integer equal to or larger than 2) storage regions each having a capacity of 256 kb. The processing section 112 performs control for supplying electric power to q (q is an integer equal to or larger than 1 and equal to or smaller than p) storage regions among the p storage regions. A value of q and a combination concerning selection of the q storage regions among the p storage regions are specified, for example, in the operation programs.

Consequently, it is possible to change a usable capacity (in the example explained above, qx256 kb) of the storing section 113 according to the operation mode. Therefore, it is possible to suppress an increase in power consumption due to operation of an excessive memory and insufficiency of a storage region and perform memory operation matched with a situation. Note that various modified implementations are possible concerning a specific configuration of the storage regions. The capacities of the storage regions are not limited to 256 kb and may be different for each of the storage regions.

In general, as processing executed in an operation program is complicated, a region necessary for loading of the operation program and a work area tend to increase. Therefore, the number of storage regions, to which electric power is supplied, is considered to be small in the clock display mode and large in the workout mode. For example, a value of q is set to a relation of the clock display mode 5 the activity meter mode 5 the workout mode.

As explained above with reference to FIG. 8, in some case, either one of the operation program (the sub-module) loaded to the RAM 1131 and the operation program SW0 stored in the ROM 1132 is selected and the processing section 112 operates according to the selected operation program. In this case, a power supply of a storage region on an unselected side may be turned off. Specifically, when the operation program (the sub-module) loaded to the RAM 1131 is executed, a power supply in the ROM region corresponding to the operation program SW0 is turned off. When the operation program SW0 is executed, a power supply of a region, to which the operation program is loaded, in the RAM 1131 is turned off. In the first embodiment, these kinds of control only have to be performed by the software SW (specifically, the processing section 112 that executes the software SW).

As shown in FIG. 7, in some case, the operation of the GNSS is unnecessary depending on an operation mode. In that case, the operation of a circuit concerning the GNSS in the first processor 110 is stopped. Specifically, power supply to at least the baseband circuit is stopped. Power supply to the RF circuit may be stopped. A control signal for stopping operation may be transmitted to the external oscillator 160, which is an external component of the first processor 110.

In the example shown in FIG. 7, the operation of the GNSS is unnecessary in the clock display mode and the activity meter mode. Therefore, by operating according to the operation programs (SW2 and SW3) corresponding to the operation modes, the processing section 112 performs control for stopping power supply to the circuit of the GNSS. On the other hand, the operation of the GNSS is necessary in the workout mode. Therefore, by operating according to the operation program SW1, the control section 112 performs control for executing the power supply to the circuit of the GNSS.

### 4.3 Communication Channel

As shown in FIG. 7, a sensor signal set as a processing target is different depending on an operation mode. Therefore, it is likely that a sensor, a sensor signal of which does not need to be acquired in the first place, is present depending on the operation mode.

Therefore, the biological information measuring device 100 (the processing section 112) may control ON/OFF of communication channels to the sensor group. The communication channels correspond to the first interface 1111, the third interface 1113, and the fourth interface 1114. The number of channels represents the number of sensors capable of communicating using the interfaces.

For example, when a maximum number of channels of the interfaces is r, the processing section 112 performs control for enabling s channels (s is an integer equal to or larger than 1 and equal to or smaller than r) among the r channels and disabling the remaining channels. For example, when the interfaces are six (r=6) channels, the processing section 112 may switch the enabled channels between three channels and six channels (s=3 or s=6).

In the example shown in FIG. 7, in the activity meter mode, the first processor 110 only has to perform communication with the pulse sensor and the acceleration sensor. That is, two channels are enough as the communication channels. Therefore, by operating according to the operation programs (SW2 and SW3) corresponding to the operation modes, the processing section 112 sets the enabled communication channels to three channels.

On the other hand, in the workout mode, it is necessary to acquire information output from the atmospheric pressure sensor, the terrestrial magnetism sensor, and the gyro sensor in addition to the pulse sensor and the acceleration sensor. The three channels are insufficient. Therefore, by operating according to the operation program SW1 corresponding to the workout mode, the processing section 112 performs control for setting the enabled communication channels to six channels.

Consequently, it is possible to switch the control of the communication channels according to the number of necessary sensor signals. It is possible to, for example, reduce power consumption by communication.

Note that the example in which the number of communication channels is switched at two stages is explained above. However, it goes without saying that the number of communication channels may be switched at three or more stages.

### 4.4 Modification of the Clock Signal Control

In FIG. 10, the example is shown in which the clock supply section 115 supplies any one clock signal among the first to third clock signals to the sections (in a narrow sense, the processing section 112) of the biological information measuring device 100. The frequency of the third clock signal based on the internal oscillator 116 is, for example, a given fixed value (26 HMz, etc.).

However, in a small and light device such as a wearable device assumed in this embodiment, a reduction in power consumption is important. For example, a reduction in power consumption in micro ampere order is required. Therefore, the first processor 110 includes, as shown in FIG. 1, a clock-frequency control section 1155 that changes, on the basis of at least one of the biological signal, the satellite signal, and the environment signal from the environment sensor 133, the clock frequency of the clock signal supplied to the processing section 112. It is possible to realize a further reduction in power consumption by flexibly changing the frequency of the clock signal according to the biological signal or the like.

Specifically, the clock-frequency control section 1155 changes the clock frequency by controlling the oscillation frequency of the internal oscillator 116. Consequently, it is possible to flexibly change the frequency of the clock signal according to the control on the internal oscillator 116. In the following explanation, a specific example of the internal oscillator 116 (a ring oscillator), an example of control for changing a frequency, and a method of suppressing occurrence of noise (occurrence of a glitch) involved in the frequency change are explained.

### 4.4.1 Configuration Example of the Internal Oscillator

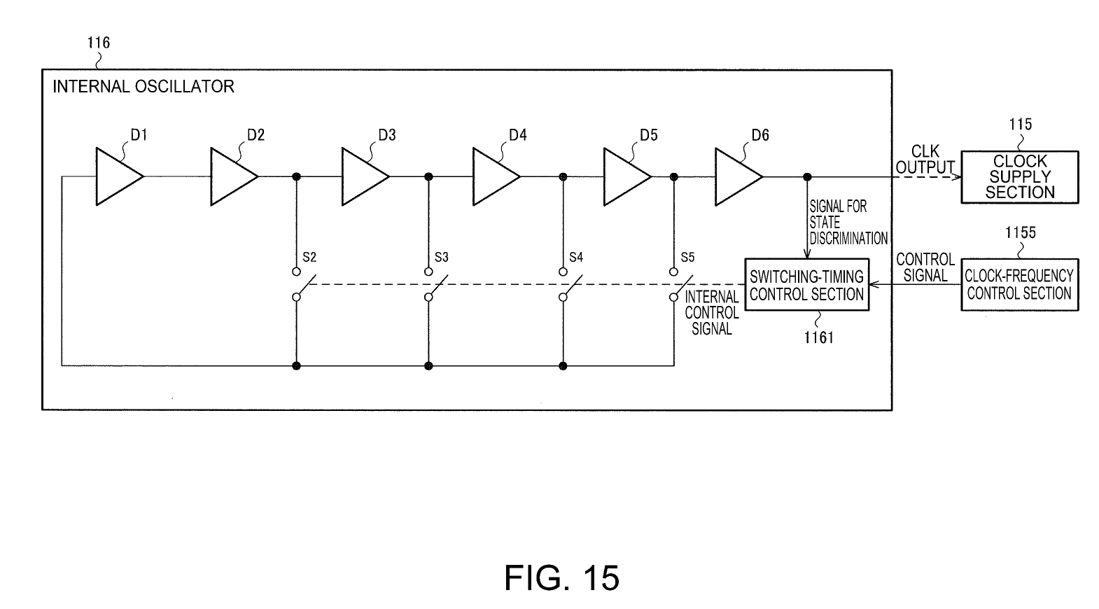

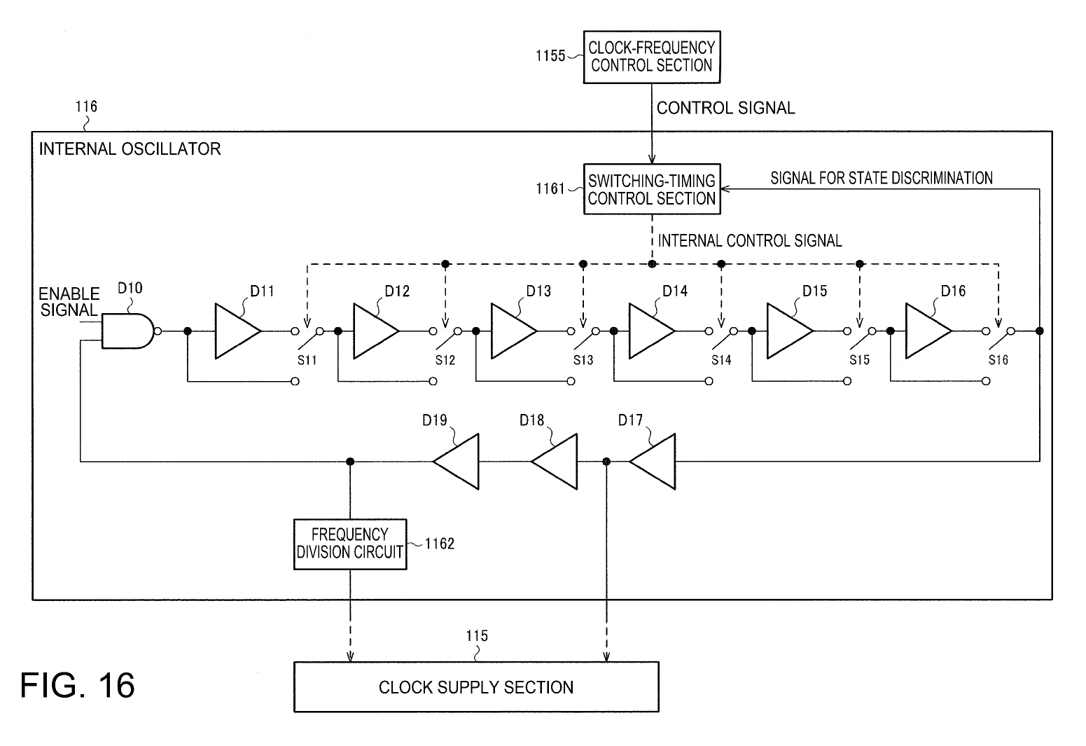

FIG. 15 is a configuration example of the internal oscillator 116 (the ring oscillator). The internal oscillator 116 includes delay elements D1 to D6, switch elements S2 to S5, and a switching-timing control section 1161. An output end of the delay element D1 is connected to an input end of the delay element D2. Concerning the delay elements D2 to D6, similarly, an output end of the delay element at the pre-stage is connected to an input end of the delay element at the next stage. The switch elements S2 to S5 are respectively provided between output ends of the delay elements D2 to D5 and an input end of the delay element D1.

The switching-timing control section 1161 receives a control signal from the clock-frequency control section 1155 and controls the oscillation frequency of the internal oscillator 116 on the basis of the control signal. Specifically, the switching-timing control section 1161 controls ON/OFF of the switch elements S2 to S5. Note that, in order to suppress occurrence of noise (a glitch), the switching-timing control section 1161 may perform processing for adjusting switching timing of a frequency (switching timing of the switch element). Details of the noise suppression are explained below.

When the switch element S2 is on and the switch elements S3 to SS5 are off, the output end of the delay element D2 is connected to the input end of the delay element D1 and the delay elements D1 and D2 are coupled in a ring shape, whereby a ring oscillator is configured. For example, the delay element D1 is a NAND circuit, the delay element D2 is an even number of (in a narrow sense, two) NOT circuits (inverters), and a not-shown enable signal is input to the delay element D1. When a delay time of signals by the delay elements D1 and D2 is represented as τ2, a cycle of a clock signal output from the internal oscillator 116 is 2×τ2 and an oscillation frequency f2 is f2=1/(2×τ2).

When the switch element S3 is on and the switch elements S2, S4, and S5 are off, the output end of the delay element D3 is connected to the input end of the delay element D1 and the delay elements D1 to D3 are coupled in a ring shape, whereby a ring oscillator is configured. When a delay time of signals by the D1 to D3 is represented as τ3, a frequency f3 of a clock signal is f3=1/(2×τ3). Since τ3>τ2, f2>f3.

Similarly, when only the switch element S4 is turned on and when only the switch element S5 is turned on, the delay time changes. Therefore, it is possible to change the frequency of the clock signal. That is, by controlling ON/OFF of the switch elements S2 to S5 (by changing the number of stages of the delay elements), it is possible to change the oscillation frequency of the internal oscillator 116. Note that the example is explained above in which the NAND circuit and the NOT circuit are used as the delay elements. However, other elements such as an operational amplifier may be used as the delay elements.

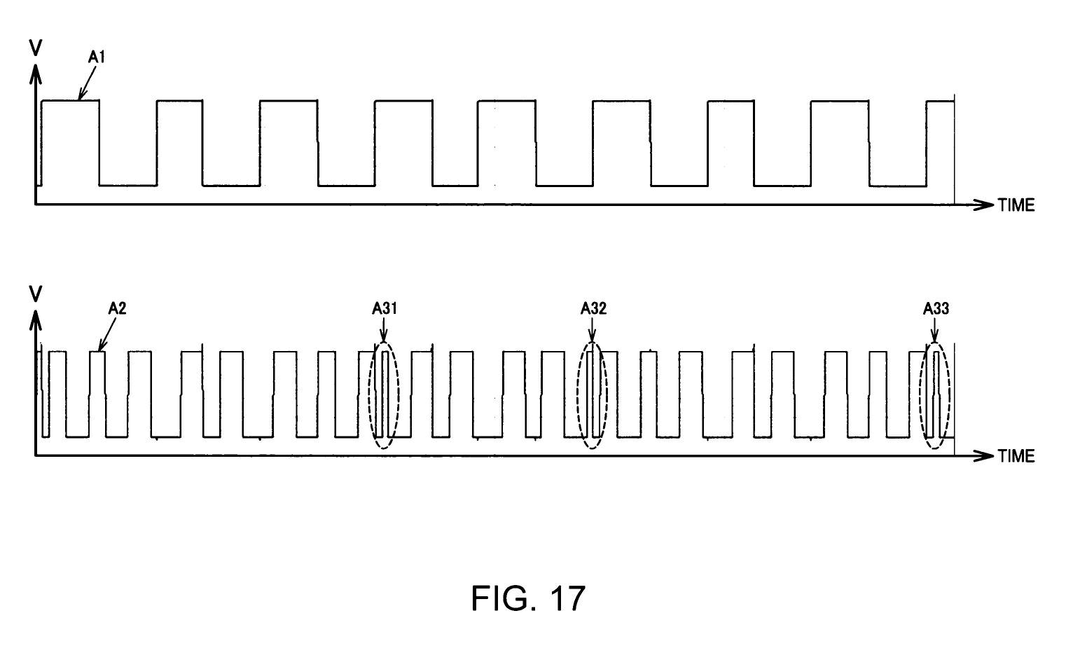

The configuration itself of the internal oscillator 116 is not limited to FIG. 15. FIG. 16 is another configuration example of the internal oscillator 116. The internal oscillator 116 shown in FIG. 16 includes delay elements D10 to D19, switch elements S11 to S16, a switching-timing control section 1161, and a frequency division circuit 1162. For example, the delay element D10 is a NAND circuit and the delay elements D11 to D19 are an even number of NOT circuits.

In the example shown in FIG. 16, at least four delay elements D10 and D17 to D19 are coupled in a ring shape, whereby a ring oscillator is configured. One end of the switch element S11 is connected to an output end or an input end of the delay element D11. When one end of the switch element S11 is connected to the output end of the delay element D11, the delay element D11 is an element forming a ring. Therefore, a delay time of a signal increases by a delay time of the delay element D11. On the other hand, when one end of the switch element S11 is connected to the input end of the delay element D1, since the delay element D11 does not form a ring, the delay time of the delay element D11 does not affect an oscillation frequency. The same applies to the switch elements S12 to S16. The switch elements S12 to S16 respectively function as switches that control whether the delay elements D12 to D16 form a ring.

When the configuration shown in FIG. 16 is used, according to the control of the switch elements S11 to S16 by the switching-timing control section 1161, it is possible to perform control for changing the delay time, that is, the oscillation frequency of the internal oscillator 116. In FIG. 16, the example is shown in which an output signal of the delay element D17 and an output signal of the frequency division circuit 1162 are output as clock signals. However, the frequency division circuit 1162 can be omitted. Alternatively, a modified implementation in which a frequency division circuit is added to FIG. 15 is also possible.

Note that, in FIGS. 15 and 16, the method of changing the oscillation frequency by changing the number of stages of the delay elements is explained. However, the internal oscillator 116 may be configured to change the oscillation frequency by suppressing a voltage.

### 4.4.2 Change Control for a Frequency

The clock-frequency control section 1155 performs control of a frequency corresponding to a situation using a biological signal or the like. Specifically, the clock-frequency control section 1155 changes a frequency of a clock signal according to an operation mode.

For example, in the clock display mode, the clock-frequency control section 1155 reduces the frequency to a minimum value (e.g., approximately 1 MHz). As shown in FIG. 7, in the clock display mode, acquisition of a biological signal and calculation may be performed at a low frequency (low accuracy). There is no problem even if a clock frequency is low.

For example, when determining that the user is in a sleeping state (a sleep state), the processing section 112 shifts to the clock display mode. The clock-frequency control section 1155 performs, while being triggered by a shift of the operation mode, control for changing the oscillation frequency of the internal oscillator 116 to be low. As the determination of the sleeping state, the processing section 112 only has to determine whether biological information may be equal to or smaller than a predetermined threshold. Before the shift to the clock display mode, it is sometimes possible to acquire an acceleration signal and a signal of the GNSS. Therefore, the processing section 112 may determine the sleeping state using a body motion signal, a satellite signal, or other environment signals. For example, when the magnitude of a body motion is equal to or smaller than a predetermined value and when determining on the basis of the satellite signal that the user is in a room, the processing section 112 determines that the user is in the sleeping state.

In the activity meter mode, the clock-frequency control section 1155 sets the frequency to an intermediate value (e.g., approximately 10 MHz). For example, when determining that the user is awake but is not performing an intense activity such as workout, the processing section 112 shifts to the activity meter mode. The clock-frequency control section 1155 performs, while being triggered by the shift of the operation mode, control for changing the oscillation frequency of the internal oscillator 116.

For example, in the clock display mode, when a value of biological information (a pulse rate) exceeds a predetermined threshold, the processing section 112 shifts to the activity meter mode. Alternatively, the processing section 112 may acquire body motion information intermittently (e.g., one per one minute) in the clock display mode and use the body motion information as well for the shift determination to the activity meter mode. When determining on the basis of the body motion information that the user is repeating a movement and a stop, the processing section 112 performs shift processing to the activity meter mode.

In the workout mode, the clock-frequency control section 1155 sets the frequency to a relatively high frequency. For example, a frequency in a normal workout mode is 16 MHz and a frequency in a high-load workout mode is 26 MHz.

Usually, in the workout mode, it is assumed that the GNSS is on and the clock signal (the first clock signal or the second clock signal) from the external oscillator 160 can be acquired. However, when indoor workout such as exercise in a gym is performed, a state in which satellite signals cannot be received continues. If a reduction in the power consumption of a wearable device is taken into account, control for stopping the reception of the satellite signals, that is, control for stopping the external oscillator 160 is sometimes performed while being triggered by a plurality of times (a predetermined time) of reception failures of the satellite signals. For example, the processing section 112 performs processing for stopping the operation of the external oscillator 160 (and the reception of satellite signals) and executing the operation intermittently (e.g., one per one minute or once per ten minutes). In this case, even in the workout mode, a clock signal based on the internal oscillator 116 is supplied to the sections of the first processor 110. That is, even in the workout mode, the control for changing the oscillation frequency of the internal oscillator 116 is sometimes necessary. By controlling the oscillation frequency of the internal oscillator 116 with the clock-frequency control section 1155, it is possible to supply a clock signal having a high frequency suitable for the workout.

The clock signal based on the internal oscillator 116 is not prevented from being used in outdoor workout. For example, when an execution frequency of positioning based on satellite signals may be low, as in the example explained above, startup of the external oscillator 160 is intermittently executed to supply the clock signal based on the internal oscillator 116 to the processing section 112 and the like. In this case as well, the control of the oscillation frequency of the internal oscillator 116 by the clock-frequency control section 1155 is important.

When the method explained above is applied to the sensor information processing device explained above, the processing section (the processing section 112) of the sensor information processing device performs, according to an operation program loaded to the storing section (e.g., the RAM 1131), processing for changing a clock frequency of a clock signal supplied to the processing section.

In the example shown in FIG. 9, the processing section 112 operates according to the application changer to load the operation program SWi corresponding to the operation mode from the nonvolatile memory (the nonvolatile memory 114). For example, association between an operation program and an oscillation frequency of the internal oscillator (the internal oscillator 116) in the operation program is defined in the application changer. The processor 112 operates according to the application changer, whereby the control processing of the internal oscillator 116 is executed such that a clock signal having a frequency corresponding to the operation program SWi serving as the load target is supplied. Specifically, the processing section 112 performs processing for instructing the clock-frequency control section 1155 to change the oscillation frequency.

As explained above, the oscillation frequency of the internal oscillator 116 is changed according to the operation mode (the operation program). However, the oscillation frequency of the internal oscillator 116 may be changed in one operation mode.

For example, in a state in which the sensor information processing device is operating in a given operation mode, the clock-frequency control section 1155 changes a frequency according to presence or absence of an interruption to the CPU (the processing section 112). Processing for calculating a pulse rate is realized by, for example, processing for performing frequency conversion (FFT: fast Fourier transform) to calculate a pulse frequency. In the FFT, pulse wave signals for a predetermined time (e.g., four seconds or sixteen seconds) are accumulated and the processing is performed targeting the accumulated data. In this example, a processing load is relatively small in an accumulation period of signals and is relatively large in a calculation period in which calculation of the FFT is actually performed. Therefore, for example, a hardware interruption is set to occur when data enough for executing the FFT are accumulated in the storing section 113. In this case, the clock-frequency control section 1155 changes the frequency to be high (e.g., 10 MHz) when an interruption occurs and changes the frequency to be low (e.g., 1 MHz) after a calculation period of the FFT ends. More specifically, the operation program is set to be capable of receiving an interruption. When an interruption occurs, as processing corresponding to the interruption, the clock-frequency control section 1155 performs processing for changing the oscillation frequency of the internal oscillator 116. Consequently, even in one operation mode, it is possible to perform control of the oscillation frequency corresponding to the processing load. It is possible to realize a further reduction in power consumption. Note that the operation mode is, for example, the activity meter mode. However, it is also possible to change the oscillation frequency of the internal oscillator 116 in the other operation modes.

### 4.4.3 Suppression of Noise Occurrence

As explained above, by changing the frequency of the clock signal (the oscillation frequency of the internal oscillator 116), it is possible to perform flexible control corresponding to a situation and realize a reduction in power consumption. However, in a circuit capable of changing a frequency, it is widely known that glitch noise occurs in an output depending on timing for switching the frequency. When a glitch occurs in the clock signal, the circuit operating with the clock signal is likely to cause malfunction.

FIG. 17 is a waveform chart for explaining that a glitch occurs because of switching control for a frequency. A1 in FIG. 17 represents a control signal for controlling the frequency. In an example shown in FIG. 17, a rising edge and a falling edge correspond to switching timings of the frequency. The control signal is a signal output from the clock-frequency control section 1155 to the internal oscillator 116. A2 in FIG. 17 represents a clock signal in the case in which the control signal is directly used for the control (e.g., in the case in which the control signal is directly supplied to a tap for switching a switch element). As indicated by A31 to A33 in FIG. 17, a glitch (a pulse having extremely narrow width) occurs depending on a state of the internal oscillator 116 during a frequency change.

In order to suppress the occurrence of the glitch, for example, it is conceivable to operate the sections of the first processor 110 in a procedure explained below. First, the processing section 112 determines whether the external oscillator 160 is started. When the external oscillator 160 is stopped, the processing section 112 starts the external oscillator 160. Subsequently, the clock supply section 115 selects the clock signal (the first clock signal or the second clock signal) based on the external oscillator 160 and supplies the selected clock signal to the sections of the first processor 110. Thereafter, the clock-frequency control section 1155 executes control for changing the oscillation frequency of the internal oscillator 116. When the oscillation frequency of the internal oscillator 116 stabilizes, the clock supply section 115 selects the clock signal (the third clock signal) based on the internal oscillator 116 and supplies the clock signal to the sections of the first processor 110. Note that, when the oscillation frequency of the internal oscillator 116 is changed, control for once stopping the operation of the internal oscillator 116 may be performed. According to the procedure explained above, a clock signal including a glitch is not supplied. Therefore, it is possible to suppress malfunction of the processing section 112 and the like.

However, in the example explained above, it is necessary to temporarily operate the external oscillator 160. Therefore, power consumption increases. Since change control for the clock signal supplied to the sections of the first processor 110, ON/OFF control of the oscillators, and the like are necessary, the control is complicated.

Taking this point into account, as shown in FIGS. 15 and 16, the internal oscillator 116 includes the switching-timing control section 1161 (a switching-timing control circuit or a logical circuit) that performs, when the clock frequency is changed, switching timing control for suppressing glitch occurrence in the clock signal.

The control signal from the clock-frequency control section 1155 does not take into account a state of the internal oscillator 116. Therefore, when the control signal is directly connected to a tap for frequency switching (a tap that switches the number of stages of the delay circuit, for example, a tap that switches ON/OFF of the switch element), a glitch is likely to occur as in the example shown in FIG. 17 depending on switching timing. In that respect, if the switching timing is appropriately controlled by the switching-timing control section 1161, it is possible to suppress the occurrence of the glitch.

If it is taken into account that it depends on the state of the internal oscillator 116 whether a glitch occurs, not only the control signal but also a signal for state discrimination representing the state of the internal oscillator 116 is input to the switching-timing control section 1161. The signal for state discrimination is an output signal of any one of the delay elements configuring the internal oscillator 116 (the ring oscillator). As shown in FIG. 15, the signal for state discrimination is a clock signal (e.g., an output signal of the delay element D6), which is an output of the internal oscillator 116. However, the signal for state discrimination may be signals of the other nodes (e.g., output signals of the delay elements D1 to D5) of the internal oscillator 116. Consequently, it is possible to determine the switching timing taking into account the state of the internal oscillator 116. Therefore, it is possible to suppress the occurrence of the glitch.

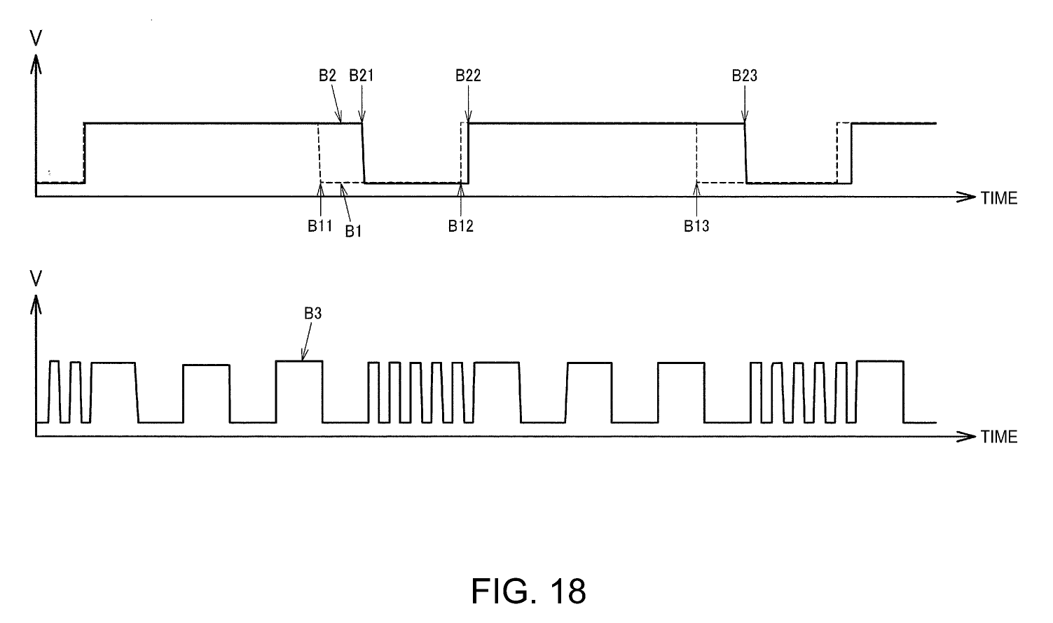

FIG. 18 is a waveform chart for explaining switching timing control for a frequency in this embodiment. In FIG. 18, B1 represents a control signal from the clock-frequency control section 1155, B2 represents a signal output by the switching-timing control section 1161 (hereinafter referred to as internal control signal), and B3 represents a clock signal controlled by the internal control signal.

As indicated by the control signal B1 shown in FIG. 18, timing when a switching instruction is given from the clock-frequency control section 1155 (a rising edge and a falling edge of the control signal) does not take into account the state of the internal oscillator 116 and, in some case, causes a glitch. In that regard, the internal control signal B2 output by the switching-timing control section 1161 does not immediately change even if the control signal B1 changes (e.g., B11 to B13) and waits until and changes at timing (e.g., B21 to B23) when a glitch does not occur. In an example of the clock signal B3 shown in FIG. 18, timing when a signal before the frequency switching is at low level (and immediately before the rising edge) and a signal after the frequency switching is at the low level (immediately before the rising edge) is set as switching timing of the frequency.

A present state (in a narrow sense, a phase and the like of the clock signal) of the internal oscillator 116 can be discriminated by the signal for state discrimination. Since a circuit configuration of the internal oscillator 116 is known, a circuit configuration before the switching and a circuit configuration after the switching are also known. By combining the circuit configuration before the switching, the circuit configuration after the switching, and the present state of the internal oscillator 116, it is possible to predict a state of the internal oscillator 116 after the frequency switching (in a narrow sense, the clock signal after the switching). The switching-timing control section 1161 determines, taking into account states of the clock signal before and after the frequency switching, appropriate switching timing when a glitch does not occur.

Note that the switching-timing control section 1161 may estimate the clock signal after the frequency switching and presence or absence of occurrence of a glitch every time. However, the switching-timing control section 1161 is not limited to this. For example, if a frequency before the switching and a frequency after the switching are determined, timing when a glitch does not occur can be calculated in advance. Specifically, the switching-timing control section 1161 is capable of estimating in advance a phase range in which a glitch does not occur even if the frequency is switched as long as a phase of the clock signal before the frequency switching is within the phase range. Therefore, for example, the storing section 113 may retain a table that associates the frequencies before and after the switching and the appropriate switching timing. The switching-timing control section 1161 may determine the appropriate switching timing referring to the table. Alternatively, the clock-frequency control section 1155 may be configured by a given logical circuit. For example, if the signal for state discrimination is appropriately selected, the internal control signal B2 can be generated by a logical operation of the signal for state discrimination and the control signal (B1). In this case, it is possible to realize the switching-timing control section 1161 with a simple logical circuit.

Note that, in a narrow sense, as shown in an example shown in FIG. 18, the switching timing is desirably set to timing when the clock signal before the switching and the clock signal after the switching are in substantially the same phase (immediately before the rising edge). However, timings when a glitch does not occur are present other than this timing. Therefore, the timings are not prevented from being set as the switching timing.

### 5. Flow of Processing

FIG. 11 is a flowchart for explaining processing of the biological information measuring device 100 (the first processor 110) in this embodiment. More specifically, processing executed by the processing section 112 on the basis of the application changer stored in the ROM 1132 is explained. Note that, as explained in the first embodiment, the processing shown in FIG. 11 may be executed on the basis of software stored in the RAM region. An example is explained below in which pulse information is used as a biological signal. However, as explained above, various expansions are possible concerning the biological signal.

When the processing is started, first, the processing section 112 (the application changer) executes hardware control at a start time (step S101). In the example shown in FIG. 11, the processing section 112 instructs the clock supply section 115 to supply the third clock signal, which is a signal output from the internal oscillator 116 (the ring oscillator). Further, the processing section 112 turns on a circuit necessary for detection of a pulse signal and a time function and turns off the GNSS and the other components.

Thereafter, the processing section 112 detects a pulse signal and performs switching control for an operation program (an application). Specifically, the processing section 112 only has to execute a pulse information detection loop in steps S102 to S110.

In the pulse information detection loop, the processing section 112 determines whether an application is stopped, that is, presence or absence of an operation program being executed (step S103). When it is determined NO in step S103, that is, an active application is present, the processing section 112 performs application end processing (step S104). Note that the application end processing includes determination concerning whether to end the application. In some case, even if the processing in step S104 is executed, the application does not end and continues operation.

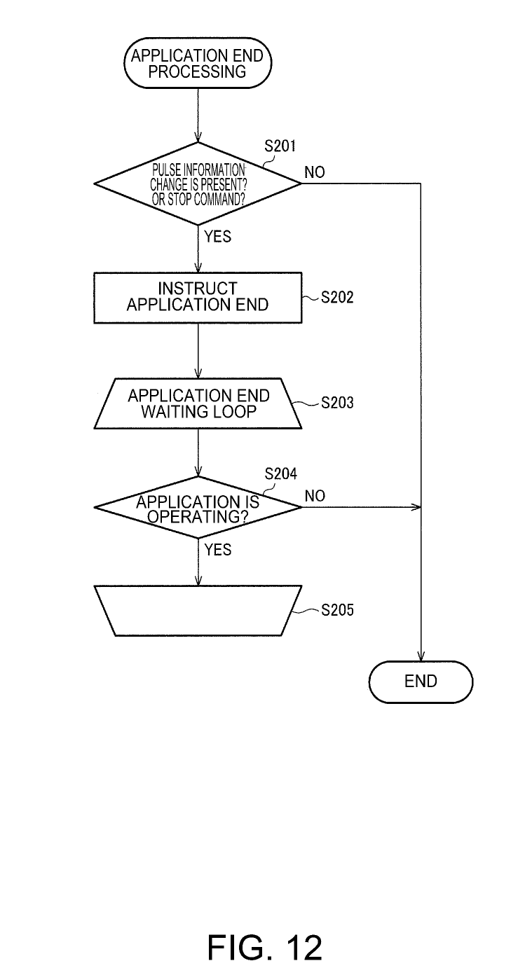

FIG. 12 is a flowchart for explaining the application end processing. When the processing is started, the processing section 112 (the application changer) determines whether a change in pulse information is present and whether a stop command is present (step S201). The change in the pulse information specifically represents a change of a degree that makes it necessary to change an operation program (an operation mode) and, in the example explained above, a change in a pulse rate exceeding the thresholds. The stop command is an instruction for stopping the operation of the application changer (in a broader sense, the entire biological information measuring device 100) and is information based on an input by the user and determination in the second processor 120 (the host CPU).

When it is determined NO in step S201, that is, a change in the pulse information is absent and the stop command is absent, the processing section 112 omits processing in steps S202 to S205 and ends the processing shown in FIG. 12. That is, in this case, the operation of the application being executed is continued.

On the other hand, when it is determined YES in step S201, that is, at least one of a change in the pulse information and the stop command is present, the processing section 112 ends the application being executed. When a change in the pulse information is present, it is necessary to switch an operation program to be executed. Therefore, as preparation for a start of a different operation program, the processing section 112 ends the operation program currently being executed. When the stop command is present, it is necessary to end the application changer after ending the application being executed. Therefore, the processing section 112 ends the operation program currently being executed as a part of operation stop processing.

Specifically, the processing section 112 instructs an application operating as another task to end (step S202). The processing section 112 continues a loop of steps S203 to S205 and stays on standby until the operation of the application ends according to the instruction. When it is determined NO in step S204, that is, the end of the application is completed, the processing section 112 leaves the loop and ends the processing shown in FIG. 12.

Referring back to FIG. 11, when it is determined YES in step S103 or after step S104, the processing section 112 determines whether the stop command is absent (step S105). When it is determined NO in step S105, that is, when the stop command is present, the processing section 112 ends the processing shown in FIG. 11. When it is determined YES in step S105, the processing section 112 determines whether a change is absent in the pulse information (step S106).

When it is determined NO in step S106, that is, when a change is present in the pulse information, the processing section 112 performs application start processing (step S107).

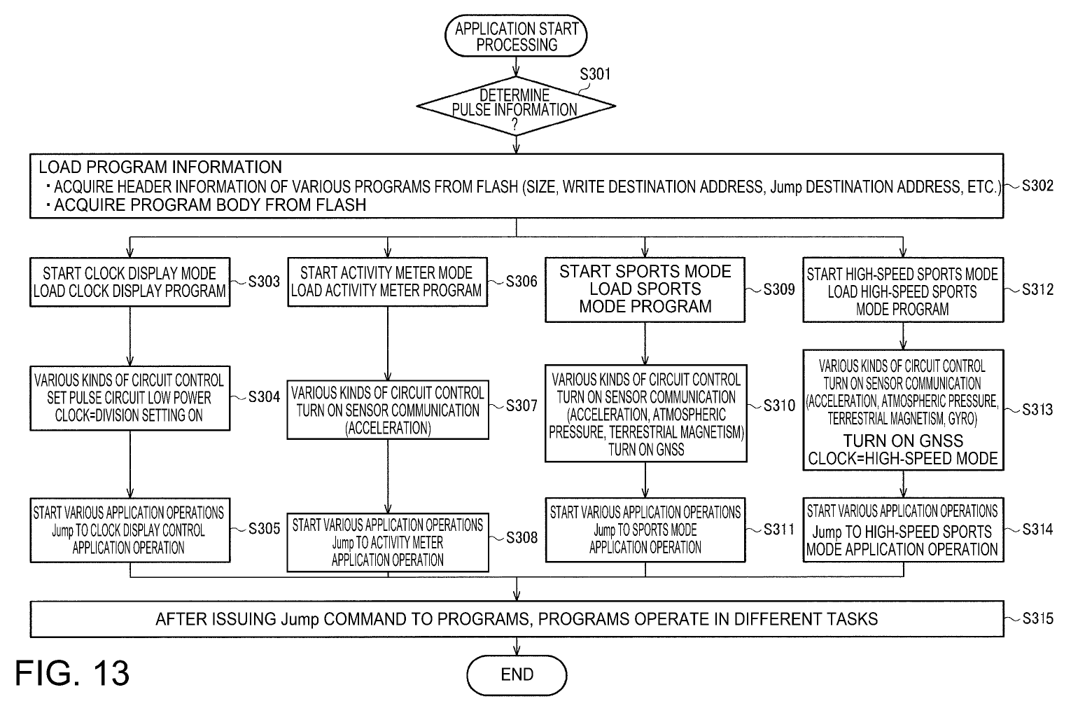

FIG. 13 is a flowchart for explaining the application start processing in step S107. When the processing is started, the processing section 112 (the application changer) determines the pulse information (step S301). This is processing for determining on the basis of the pulse information which operation mode (operation program or application) is selected. In the example explained above, the processing section 112 performs comparison processing of the pulse rate and the thresholds.

The processing section 112 loads program information of an operation program corresponding to the selected operation mode. Specifically, the processing section 112 acquires header information of the operation programs from the nonvolatile memory 114 (step S302).

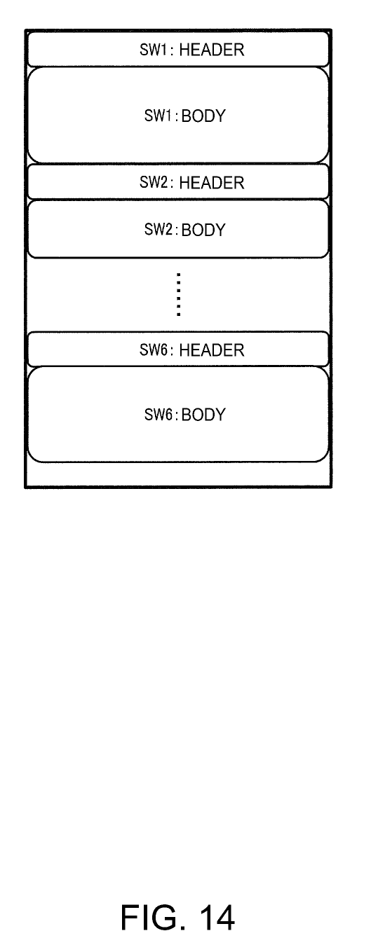

FIG. 14 is a diagram for explaining an operation program retaining format of the nonvolatile memory 114. In the nonvolatile memory 114, the header information and bodies of the operation programs are alternately stored. When there are six operation programs SW1 to SW6 as the operation programs, first, header information of the operation program SW1 is stored and a body of the operation program SW1 is stored in a storage region following the header information. Header information of the operation program SW2 is stored in a storage region following the body of the operation program SW1. Subsequently, similarly, header information and operation program bodies of the operation programs SW3 to SW6 are arranged.

In the header information, size information of the operation program body, write destination address information in the RAM 1131, and jump destination address information are stored. Note that the jump destination address information is an address indicating an execution start position of the operation program. Since size information is included in the header information, by using a configuration shown in FIG. 14, header positions of the operation programs are clarified. For example, if the header information of the operation program SW1 is read, a size of the operation program SW1 body is known. Therefore, it is possible to learn a storage region of the header information of the operation program SW2. However, FIG. 14 is an example of a format for retaining the operation programs. Other formats are not prevented from being used.

The processing section 112 reads out, on the basis of the header information, operation program bodies corresponding to the header information from the nonvolatile memory 114 (step S302).

Thereafter, the processing section 112 executes operations in the operation modes on the basis of the operation programs loaded to the RAM 1131. For example, in the clock display mode, the processing section 112 loads the operation program corresponding to the clock display mode read out in step S302 to the RAM 1131 (step S303) and performs the various kinds of circuit control as explained above (step S304). In the clock display mode, since time information and minimum pulse information only have to be able to be acquired, the processing section 112 executes control for dividing a clock signal. When the circuit control ends, the processing section 112 executes the operation program from a predetermined address on the basis of jump destination address information in the header information to start operation of a clock display application (step S305). After the start of the application, the processing section 112 operates the application as a task different from the application changer (step S315).

The same applies to the other operation modes. The processing section 112 loads the operation programs (steps S306, S309, and S312), performs the circuit control (steps S307, S310, and S313), and starts operations of applications on the basis of jump destination address information (steps S308, S311, and S314). Note that, in FIG. 13, an example is shown in which two modes, that is, a sports mode (a normal workout mode) and a high-speed sports mode (a high-load workout mode) can be set as the workout mode.

In the activity meter mode, the processing section 112 performs control for turning on a communication channel for acquiring an acceleration signal as the circuit control.

In the sports mode, the processing section 112 performs control for turning on communication channels for acquiring an acceleration signal, an atmospheric pressure signal, and a terrestrial magnetism signal as the circuit control. The processing section 112 performs control for turning on power supplies of the sections related to the GNSS.

In the high-speed sports mode, the processing section 112 performs control for turning on communication channels for acquiring an acceleration signal, an atmospheric pressure signal, a terrestrial magnetism signal, and a gyro signal as the circuit control. The processing section 112 turns on the power supplies of the sections related to the GNSS. The processing section 112 may perform control for using a second clock signal having a relatively high frequency as the clock signal.

In this embodiment, control of the clock frequency corresponding to the operation mode may be performed by the clock-frequency control section 1155. The control of the clock frequency is executed, for example, in the circuit control (steps S304, S307, S310, and S313) before the starts of the operations of the applications. In step S304, the processing section 112 instructs the clock-frequency control section 1155 to perform control for changing the oscillation frequency of the internal oscillator 116 to a relatively low frequency. In step S307, the processing section 112 instructs the clock-frequency control section 1155 to perform control for changing the oscillation frequency of the internal oscillator 116 to an intermediate frequency. In steps S310 and S313, the processing section 112 instructs the clock-frequency control section 1155 to perform control for changing the oscillation frequency of the internal oscillator 116 to a relatively high frequency. As an example, the frequency set in step S304 is 1 MHz, the frequency set in step S307 is 10 MHz, the frequency set in step S310 is 16 MHz, and the frequency set in step S313 is 26 MHz.

In this embodiment, as a task different from the application changer, change control for the clock frequency by the clock-frequency control section 1155 may be performed in the application that starts the operation in step S315. For example, as in the example explained above, a hardware interruption is waited in the application started to be executed in S315. When an interruption is detected, the control for changing the clock frequency is performed.

Referring back to FIG. 11, after the end of the application start processing (step S107) shown in FIG. 13, the processing section 112 shifts to a halt state and waits for a timer interruption (step S108). When the interruption is executed, the processing section 112 returns from the halt state (step S109) and continues a pulse wave detection loop (returns from step S110 to step S102). When it is determined YES in step S106, the processing section 112 skips the application start processing and shifts to step S108.

The embodiments applied with the invention and the modifications of the embodiments are explained above. However, the invention is not limited to the embodiments and the modifications per se. In an implementation stage, the constituent elements can be modified and embodied in a range not departing from the spirit of the invention. Various inventions can be formed by appropriately combining the plurality of constituent elements disclosed in the embodiments and the modifications. For example, several constituent elements may be deleted from all the constituent elements described in the embodiments and the modifications. Further, the constituent elements described in the different embodiments and modifications may be combined as appropriate. The terms described together with broader-sense or synonymous different terms at least once in the specification or the drawings can be replaced with the different terms in any part of the specification or the drawings. In this way, various modifications and applications are possible within a range not departing from the spirit of the invention.

What is claimed is:

1. A biological information measuring device comprising:
a first processor including a first interface configured to acquire a biological signal output from a biological sensor and a second interface configured to acquire a satellite signal; and
a second processor configured to control at least one of a display section and a communication section and electrically connected to the first processor.

2. The biological information measuring device according to claim 1, wherein
the first processor includes a processing section connected to the first interface and the second interface and configured to perform processing based on the biological signal and the satellite signal.

3. The biological information measuring device according to claim 2, wherein
the first processor is capable of operating in any one operation mode of a plurality of operation modes, and
the processing section performs, on the basis of the biological signal, processing for switching an operation mode among the plurality of operation modes.

4. The biological information measuring device according to claim 2, wherein
the first processor includes a third interface configured to acquire a body motion signal output from a body motion sensor, and
the processing section performs, on the basis of at least one of the biological signal and the body motion signal, processing for switching an operation mode among the plurality of operation modes.

5. The biological information measuring device according to claim 3, wherein
the first processor includes a nonvolatile memory configured to store a plurality of operation programs each corresponding to a respective one of the plurality of operation modes.

6. The biological information measuring device according to claim 5, wherein
the processing section, the first interface, and the second interface are formed on a semiconductor chip of one chip, and
the nonvolatile memory is stacked on the semiconductor chip.

7. The biological information measuring device according to claim 3, wherein
the plurality of operation modes include at least two of a clock display mode, an activity meter mode, and a workout mode.

8. The biological information measuring device according to claim 1, wherein
the first processor includes a fourth interface configured to acquire an environment signal output from an environment sensor.

9. The biological information measuring device according to claim 1, wherein
the first processor further includes a clock supply section configured to receive an input of a plurality of clock signals and supply a selected clock signal among the plurality of clock signals, and
the clock supply section selects, on the basis of the biological signal, a clock signal from among the plurality of clock signals to be supplied.

10. The biological information measuring device according to claim 9, wherein
the clock supply section performs selection of a clock signal among the plurality of clock signals to be supplied at a start time and selection of a clock signal among the plurality of clock signals to be supplied at an operation time after the start.

11. The biological information measuring device according to claim 9, further comprising an external oscillator provided on an outside of the first processor, wherein
the first processor includes an internal oscillator, and
the clock signals include a signal output from the external oscillator and a signal output from the internal oscillator.

12. The biological information measuring device according to claim 2, wherein
the first processor includes a clock-frequency control section configured to change, on the basis of at least one of the biological signal, the satellite signal, and an environment signal output from an environment sensor, a clock frequency of a clock signal supplied to the processing section.

13. The biological information measuring device according to claim 12, wherein
the first processor includes an internal oscillator configured to generate the clock signal, and
the clock-frequency control section controls an oscillation frequency of the internal oscillator to change the clock frequency.

14. The biological information measuring device according to claim 13, wherein
the internal oscillator includes a switching-timing control section configured to perform, when the clock frequency is changed, switching timing control for suppressing glitch occurrence in the clock signal.

15. A wearable device comprising the biological information measuring device according to claim 1.

16. A sensor information processing device comprising:
a processing section;
an interface configured to acquire a biological signal from a biological sensor;
a nonvolatile memory configured to store a plurality of operation programs each corresponding to a respective one of a plurality of operation modes; and
a storing section, wherein
each operation program is a computer program that operates on the basis of sensor information output from the biological sensor,
an operation program selected out of the plurality of operation programs is loaded to the storing section, and
the processing section operates according to the operation program loaded to the storing section.

17. The sensor information processing device according to claim 16, wherein
the processing section performs, according to the operation program loaded to the storing section, processing for changing a clock frequency of a clock signal supplied to the processing section.

18. A biological information measuring device comprising:
a first processor that includes a first interface and a second interface, the first interface acquiring a biological signal output from a biological sensor, the second interface acquiring a satellite signal; and
a second processor that controls at least one of (1) a display and (2) a communication interface and an antenna, and is electrically connected to the first processor.

19. The biological information measuring device according to claim 18, further comprising the biological sensor.
